(19) United States
(12) Patent Application Publication
(10) Pub. No.: US 2024/0025949 A1
(43) Pub. Date: Jan. 25, 2024

(54) Beta-Arrestin Effectors and Compositions and Methods of Use Thereof

(71) Applicant: Trevena, Inc., Chesterbrook, PA (US)

(72) Inventor: Mark Demitrack, Chesterbrook, PA (US)

(21) Appl. No.: 17/936,956

(22) Filed: Sep. 30, 2022

Related U.S. Application Data
(60) Provisional application No. 63/261,838, filed on Sep. 30, 2021.

(51) Int. Cl. C07K 7/64; A61P 11/00; A61P 7/02

(52) U.S. Cl. CPC C07K 7/64; A61P 11/00; A61P 7/02

(57) ABSTRACT

This application describes a family of compounds acting as beta-arrestin effectors that can be used, for example, for treating acute respiratory distress syndrome, for preventing and treating thrombosis, platelet adhesion, and platelet aggregation, and/or for reducing a D-Dimer response, in a subject with ARDS or a viral infection, such as a coronavirus infection.

### CROSS-REFERENCE TO RELATED APPLICATIONS

This application claims priority to U.S. Provisional Application No. 63/261,838, filed Sep. 30, 2021, U.S. Provisional Application No. 63/013,061, filed Apr. 21, 2020, and U.S. Provisional Application No. 63/049,826, filed Jul. 9, 2020, each of which is hereby incorporated by reference in its entirety.

This application also relates to U.S. Pat. Nos. 8,486,885, 8,796,204, 8,809,260, 8,946,142, 8,993,511, 9,518,086, 9,534,017, and 9,611,293, each of which is incorporated herein by reference in its entirety.

### FIELD

This application relates to a family of compounds acting as β-arrestin effectors. Such compounds can be used, for example, in the treatment of acute respiratory distress syndrome (ARDS), in the prevention and treatment of thrombosis, platelet adhesion, platelet aggregation, and related disorders, and/or in the reduction of a D-Dimer response in a subject with ARDS or a viral infection.

### BACKGROUND

Drugs targeting GPCRs have been developed based on a signaling paradigm in which stimulation of the receptor by an agonist (e.g., angiotensin II) leads to activation of a heterotrimeric “G protein,” which then leads to second messenger/down-stream signaling (e.g., via diacylglycerol, inositol-triphosphate, calcium, etc. . . . ) and changes in physiological function (e.g., blood pressure and fluid homeostasis). There is a need for additional drugs that target GPCRs for the treatment of pathology associated with blood pressure and fluid homeostasis.

The foregoing description of the related art is not intended in any way as an admission that any of the documents described therein, including pending United States patent applications, are prior art. Moreover, the description herein of any disadvantages associated with the described products, methods, and/or apparatus is not intended to limit the embodiments. Indeed, aspects of the embodiments may include certain features of the described products, methods, and/or apparatus without suffering from their described disadvantages.

### SUMMARY

According to some embodiments, the present embodiments provides for novel β-arrestin effectors having the formula of: Xx-Yy-Val-Ww-Zz-Aa-Bb-Cc, which can be used for, amongst other things, treating acute respiratory distress syndrome (ARDS), preventing or treating thrombosis, platelet adhesion, platelet aggregation, and related disorders, and/or reducing a D-Dimer response in a subject with ARDS or a viral infection.

In the formula above, variables Aa, Bb, Cc, Ww, Xx, Yy, and Zz can be selected from the respective groups of chemical or biological moieties later described in the detailed description. β-arrestin effector derivatives and mimetics are also provided. Also provided are processes for preparing the compounds that can be used in the provided methods and uses.

According to some embodiments, the present embodiments provide for β-arrestin effectors having the structure:

Xx-Arg-Val-Ww-Zz-His-Pro-Cc;

or pharmaceutically acceptable salt, solvate, or hydrate thereof. In the structure above, variables Cc, Ww, Xx, and Zz can be selected from the respective groups of chemical or biological moieties later described in the detailed description. 0-arrestin effector derivatives and mimetics are also provided.

According to some embodiments, the compounds have the following formula:

Sar-Arg-Val-Ww-Zz-His-Pro-Cc

or pharmaceutically acceptable salt, solvate, or hydrate thereof. In the structure above, variables Cc, Ww, and Zz can be selected from the respective groups of chemical or biological moieties later described in the detailed description. β-arrestin effector derivatives and mimetics are also provided.

According to some embodiments, the compounds have the formula:

Sar-Arg-Val-Ww-OMTh-His-Pro-Cc,

or pharmaceutically acceptable salt, solvate, or hydrate thereof. In the structure above, variables Cc, and Ww can be selected from the respective groups of chemical or biological moieties later described in the detailed description. β-arrestin effector derivatives and mimetics are also provided.

According to some embodiments, the peptide or peptide mimetic are selected from the group consisting of
a) a peptide or peptide mimetic comprising the sequence of

Xx-Yy-Val-Ww-Zz-Aa-Bb-Cc,
wherein Xx is selected from the group consisting of null, sarcosine, N-methyl-L-alanine, N-methyl-D-alanine, N,N-dimethylglycine, L-aspartic acid, D-aspartic acid, L-glutamic acid, D-glutamic acid, N-methyl-L-aspartic acid, N-methyl-L-glutamic acid, pyrrolid-1-ylacetic acid, and morpholin-4-ylacetic acid;
Yy is selected from the group consisting of L-arginine and L-lysine;
Ww is selected from the group consisting of L-isoleucine, glycine, L-tyrosine, O-methyl-L-tyrosine, L-valine, L-phenylalanine, 3-hydroxy-L-tyrosine, 2,6-dimethyl-L-tyrosine, 3-fluoro-L-tyrosine, 4-fluorophenyl-L-alanine, 2,6-difluoro-L-tyrosine, 3-nitro-L-tyrosine, 3,5-dinitro-L-tyrosine, 3,5-dibromo-L-tyrosine, 3-chloro-L-tyrosine, O-allyl-L-tyrosine, and 3,5-diiodo-L-tyrosine;
Zz is selected from the group consisting of L-isoleucine, L-valine, L-tyrosine, L-glutamic acid, L-phenylalanine, L-histidine, L-lysine, L-arginine, O-methyl-L-threonine, D-alanine, and L-norvaline;
Aa is selected from the group consisting of L-histidine, L-histidine-amide, and L-lysine;
Bb is selected from the group consisting of L-proline, L-proline-amide, D-proline, and D-proline-amide; and
Cc is selected from the group consisting of null, L-isoleucine, L-isoleucine-amide, glycine, glycine-amide, L-alanine, L-alanine-amide, D-alanine, D-phenylalanine, L-norvaline;
provided that when Xx is L-Aspartic acid, Cc is not L-phenylalanine; when Xx is sarcosine, Cc is not L-isoleucine; when Ww is glycine, Cc is not glycine; when Xx is sarcosine, and Zz is L-valine, Cc is not L-alanine; and when Xx is sarcosine, Ww is L-tyrosine, and Zz is L-isoleucine, Cc is not L-alanine;
b) a peptide or peptide mimetic wherein the members of the sequence of the peptide or peptide mimetic maintain their relative positions as they appear in the sequence described in (a), wherein spacers of between 1 and 3 amino acids or amino acid analogues are inserted between one or more of the amino acids or amino acid analogues as described in (a) and wherein the total length of the peptide or peptide mimetic is between 6 and 25 amino acids and/or amino acid analogues; and
c) a peptide or peptide mimetic that is at least 70% identical to the peptide or peptide mimetics described in (a).

According to other embodiments, a peptide or peptide mimetic selected from the group consisting of
a) a peptide or peptide mimetic comprising the sequence of

Xx-Arg-Val-Ww-Zz-His-Pro-Cc,
wherein Xx is selected from the group consisting of sarcosine, N-methyl-L-alanine, N-methyl-D-alanine, N,N-dimethylglycine, L-aspartic acid, D-aspartic acid, L-glutamic acid, D-glutamic acid, N-methyl-L-aspartic acid, N-methyl-L-glutamic acid, pyrrolid-1-ylacetic acid, and morpholin-4-ylacetic acid;
Ww is selected from the group consisting of L-isoleucine, glycine, L-tyrosine, O-methyl-L-tyrosine, L-valine, L-phenylalanine, 3-hydroxy-L-tyrosine, 2,6-dimethyl-L-tyrosine, 3-fluoro-L-tyrosine, 4-fluorophenyl-L-alanine, 2,6-difluoro-L-tyrosine, 3-nitro-L-tyrosine, 3,5-dinitro-L-tyrosine, 3,5-dibromo-L-tyrosine, 3-chloro-L-tyrosine, O-allyl-L-tyrosine, and 3,5-diiodo-L-tyrosine;
Zz is selected from the group consisting of L-isoleucine, L-valine, L-tyrosine, L-glutamic acid, L-phenylalanine, L-histidine, L-lysine, L-arginine, O-methyl-L-threonine, D-alanine, and L-norvaline; and
Cc is selected from the group consisting of L-isoleucine, L-isoleucine-amide, glycine, glycine-amide, L-alanine, L-alanine-amide, D-alanine, D-phenylalanine, and L-norvaline;
provided that when Xx is L-Aspartic acid, Cc is not L-phenylalanine; when Xx is sarcosine, Cc is not L-isoleucine; when Ww is glycine, Cc is not glycine; when Xx is sarcosine, and Zz is L-valine, Cc is not L-alanine; and when Xx is sarcosine, Ww is L-tyrosine, and Zz is L-isoleucine, Cc is not L-alanine;
b) a peptide or peptide mimetic wherein the members of the sequence of the peptide or peptide mimetic maintain their relative positions as they appear in the sequence described in (a), wherein spacers of between 1 and 3 amino acids or amino acid analogues are inserted between one or more of the amino acids or amino acid analogues as described in (a) and wherein the total length of the peptide or peptide mimetic is between 8 and 25 amino acids and/or amino acid analogues; and
c) a peptide or peptide mimetic that is at least 70% identical to the peptide or peptide mimetics described in (a) is provided.

According to other embodiments, the peptide or peptide mimetic is selected from the group consisting of
a) a peptide or peptide mimetic comprising the sequence of

Sar-Arg-Val-Ww-Zz-His-Pro-Cc,
wherein Ww is selected from the group consisting of L-tyrosine, 3-hydroxy-L-tyrosine, 3-fluoro-L-tyrosine, 2,6-difluoro-L-tyrosine, 3-nitro-L-tyrosine, 3,5-dinitro-L-tyrosine, and 3-chloro-L-tyrosine;
Zz is selected from the group consisting of L-isoleucine, L-lysine, and O-Methyl-L-threonine; and
Cc is selected from the group consisting of D-alanine, and L-alanine
provided that when Xx is sarcosine, Ww is L-tyrosine, and Zz is L-isoleucine, Cc is not L-alanine;
b) a peptide or peptide mimetic wherein the members of the sequence of the peptide or peptide mimetic maintain their relative positions as they appear in the sequence described in (a), wherein spacers of between 1 and 3 amino acids or amino acid analogues are inserted between one or more of the amino acids or amino acid analogues as described in (a) and wherein the total length of the peptide or peptide mimetic is between 8 and 25 amino acids and/or amino acid analogues; and
c) a peptide or peptide mimetic that is at least 70% identical to the peptide or peptide mimetics described in (a).

In some embodiments, Ww is selected from the group consisting of L-tyrosine and 3-hydroxy-L-tyrosine; and Zz is selected from the group consisting of L-isoleucine and L-lysine. In some embodiments, the peptide or peptide mimetic comprises the sequence of SEQ ID NO:23, 27 or 67.

According to other embodiments, the peptide or peptide mimetic is selected from the group consisting of
a) a peptide or peptide mimetic comprising the sequence of

Sar-Arg-Val-Ww-OMTh-His-Pro-Cc,
wherein Ww is selected from the group consisting of L-tyrosine, 3-hydroxy-L-tyrosine; 3-fluoro-L-tyrosine, and 3-chloro-L-tyrosine; and
Cc is selected from the group consisting of D-alanine and L-alanine;
b) a peptide or peptide mimetic wherein the members of the sequence of the peptide or peptide mimetic maintain their relative positions as they appear in the sequence described in (a), wherein spacers of between 1 and 3 amino acids or amino acid analogues are inserted between one or more of the amino acids or amino acid analogues as described in (a) and wherein the total length of the peptide or peptide mimetic is between 8 and 25 amino acids and/or amino acid analogues; and
c) a peptide or peptide mimetic that is at least 70% identical to the peptide or peptide mimetics described in (a).

In some embodiments, Ww is selected from the group consisting of 3-hydroxy-L-tyrosine; 3-fluoro-L-tyrosine, and 3-chloro-L-tyrosine; and Cc is L-alanine. Optionally, the peptide or peptide mimetic comprises the sequence of SEQ ID NO:77, 78 or 79.

In some embodiments, the peptide or peptide mimetic is selected from the group consisting of
a) a peptide or peptide mimetic comprising the sequence of Sar-Arg-Val-Ww-Tyr-His-Pro-NH₂,
   wherein Ww is selected from the group consisting of L-tyrosine, 3-hydroxy-L-tyrosine, 3-fluoro-L-tyrosine, 2,6-difluoro-L-tyrosine, 3-nitro-L-tyrosine, 3,5-dinitro-L-tyrosine, and 3-chloro-L-tyrosine;
b) a peptide or peptide mimetic wherein the members of the sequence of the peptide or peptide mimetic maintain their relative positions as they appear in the sequence described in (a), wherein spacers of between 1 and 3 amino acids or amino acid analogues are inserted between one or more of the amino acids or amino acid analogues as described in (a) and wherein the total length of the peptide or peptide mimetic is between 7 and 25 amino acids and/or amino acid analogues; and
c) a peptide or peptide mimetic that is at least 70% identical to the peptide or peptide mimetics described in (a).

In some embodiments, the peptide or peptide mimetic comprises the sequence of SEQ ID NO:26.

In some embodiments, a peptide or peptide mimetic is selected from the group consisting of
a) a peptide or peptide mimetic comprising the sequence of NMAla-Arg-Val-Ww-Zz-His-Pro-Cc,
   wherein Ww is selected from the group consisting of L-tyrosine, 3-hydroxy-L-tyrosine, 3-fluoro-L-tyrosine, 2,6-difluoro-L-tyrosine, 3-nitro-L-tyrosine, 3,5-dinitro-L-tyrosine, and 3-chloro-L-tyrosine;
   Zz is selected from the group consisting of L-isoleucine, L-lysine, and O-Methyl-L-threonine;
   and Cc is selected from the group consisting of D-alanine, and L-alanine;
b) a peptide or peptide mimetic wherein the members of the sequence of the peptide or peptide mimetic maintain their relative positions as they appear in the sequence described in (a), wherein spacers of between 1 and 3 amino acids or amino acid analogues are inserted between one or more of the amino acids or amino acid analogues as described in (a) and wherein the total length of the peptide or peptide mimetic is between 8 and 25 amino acids and/or amino acid analogues; and
c) a peptide or peptide mimetic that is at least 70% identical to the peptide or peptide mimetics described in (a).

In more specific embodiments, Ww is L-tyrosine, Zz is L-isoleucine, and/or Cc is L-alanine. Optionally, the peptide or peptide mimetic comprises the sequence of SEQ ID NO 34.

In some embodiments, any of the above-described embodiments are cyclic, dimerized and/or trimerized peptides or mimetics thereof.

In some embodiments, pharmaceutical compositions are provided that comprise one or more of the peptides or peptide mimetics described herein. In some embodiments, the composition comprises a pharmaceutically acceptable carrier. In some embodiments, the pharmaceutically acceptable carrier is pure sterile water, phosphate buffered saline, or an aqueous glucose solution, or any combination thereof.

In some embodiments, methods of treating acute respiratory distress syndrome (ARDS) are provided. In some embodiments, the ARDS is viral-induced ARDS. In some embodiments, the ARDS is induced by an influenza virus or a coronavirus. In some embodiments, the ARDS is caused by COVID-19 infection. In some embodiments, the ARDS is in a subject who has a viral infection. In some embodiments, the viral infection is an influenza viral infection. In some embodiments, the viral infection is a coronavirus infection. In some embodiments, the coronavirus infection is a COVID-19 infection. In some embodiments, the subject having the infection is a subject in need thereof.

In some embodiments, pharmaceutical compositions comprising a compound (for example, peptide or mimetic thereof) a pharmaceutically acceptable carrier are provided. In some embodiments, the compounds can be employed in any form, such as a solid or solution (e.g., aqueous solution) as is provided herein. The compound, for example, can be also obtained and employed in Lyophilized form alone or with suitable additives.

### DETAILED DESCRIPTION

Before the present proteins, nucleotide sequences, peptides, etc., and methods are described, it is understood that these embodiments are not limited to the particular methodology, protocols, cell lines, vectors, and reagents described, as these may vary. It also is to be understood that the terminology used herein is for the purpose of describing particular embodiments only, and is not intended to limit the scope of the present embodiments.

This application describes a family of compounds, β-arrestin effectors, with a unique profile. The compounds (peptides or peptide mimetics) can act as agonists of β-arrestin/GRK-mediated signal transduction via the AT1 angiotensin receptor. Thus, these compounds stimulate signaling pathways that provide significant therapeutic benefits in the treatment of cardiovascular diseases such as acute heart failure or an acute hypertensive crisis.

According to some embodiments, the compounds comprise the following formula:

Xx-Yy-Val-Ww-Zz-Aa-Bb-Cc,

wherein Xx is null, sarcosine, N-methyl-L-alanine, N-methyl-D-alanine, N,N-dimethylglycine, L-aspartic acid, D-aspartic acid, L-glutamic acid, D-glutamic acid, N-methyl-L-aspartic acid, N-methyl-L-glutamic acid, pyrrolid-1-ylacetic acid, or morpholin-4-ylacetic acid; Yy is L-arginine, or L-lysine; Ww is L-isoleucine, glycine, L-tyrosine, O-methyl-L-tyrosine, L-valine, L-phenylalanine, 3-hydroxy-L-tyrosine, 2,6-dimethyl-L-tyrosine, 3-fluoro-L-tyrosine, 4-fluorophenyl-L-alanine, 2,6-difluoro-L-tyrosine, 3-nitro-L-tyrosine, 3,5-dinitro-L-tyrosine, 3,5-dibromo-L-tyrosine, 3-chloro-L-tyrosine, O-allyl-L-tyrosine, or 3,5-diiodo-L-tyrosine; Zz is L-isoleucine, L-valine, L-tyrosine, L-glutamic acid, L-phenylalanine, L-histidine, L-lysine, L-arginine, O-methyl-L-threonine, D-alanine, or L-norvaline; Aa is L-histidine, L-histidine-amide, or L-lysine; Bb is L-proline, L-proline-amide, D-proline, D-proline-amide and Cc is null, L-isoleucine, L-isoleucine-amide, glycine, glycine-amide, L-alanine, L-alanine-amide, D-alanine, D-phenylalanine, L-norvaline;
provided that when Xx is L-aspartic acid, Cc is not L-phenylalanine;
provided that when Xx is sarcosine, Cc is not L-isoleucine;
provided that when Ww is glycine, Cc is not glycine;
provided that when Xx is sarcosine, and Zz is L-valine, Cc is not L-alanine;
provided that when Xx is sarcosine, Ww is L-tyrosine, and Zz is L-isoleucine, Cc is not L-alanine.

According to some embodiments, the compounds comprise the following formula:

Xx-Arg-Val-Ww-Zz-His-Pro-Cc,

wherein Xx is sarcosine, N-methyl-L-alanine, N-methyl-D-alanine, N,N-dimethylglycine, L-aspartic acid, D-aspartic acid, L-glutamic acid, D-glutamic acid, N-methyl-L-aspartic acid, N-methyl-L-glutamic acid, pyrrolid-1-ylacetic acid, or morpholin-4-ylacetic acid; Ww is L-isoleucine, glycine, L-tyrosine, O-methyl-L-tyrosine, L-valine, L-phenylalanine, 3-hydroxy-L-tyrosine, 2,6-dimethyl-L-tyrosine, 3-fluoro-L-tyrosine, 4-fluorophenyl-L-alanine, 2,6-difluoro-L-tyrosine, 3-nitro-L-tyrosine, 3,5-dinitro-L-tyrosine, 3,5-dibromo-L-tyrosine, 3-chloro-L-tyrosine, O-allyl-L-tyrosine, or 3,5-diiodo-L-tyrosine; Zz is L-isoleucine, L-valine, L-tyrosine, L-glutamic acid, L-phenylalanine, L-histidine, L-lysine, L-arginine, O-methyl-L-threonine, D-alanine, or L-norvaline; and Cc is L-isoleucine, L-isoleucine-amide, glycine, glycine-amide, L-alanine, L-alanine-amide, D-alanine, D-phenylalanine, L-norvaline.
provided that when Xx is L-aspartic acid, Cc is not L-phenylalanine;
provided that when Xx is sarcosine, Cc is not L-isoleucine;
provided that when Ww is glycine, Cc is not glycine;
provided that when Xx is sarcosine, and Zz is L-valine, Cc is not L-alanine;
provided that when Xx is sarcosine, Ww is L-tyrosine, and Zz is L-isoleucine, Cc is not L-alanine.

According to some embodiments, the compounds comprise the following formula:

Sar-Arg-Val-Ww-Zz-His-Pro-Cc,

wherein Ww is L-tyrosine, 3-hydroxy-L-tyrosine, 3-fluoro-L-tyrosine, 2,6-difluoro-L-tyrosine, 3-nitro-L-tyrosine, 3,5-dinitro-L-tyrosine, 3-chloro-L-tyrosine; Zz is L-valine or L-isoleucine; and Cc is D-alanine.

According to some embodiments, the compounds have the following formula:

Sar-Arg-Val-Ww-OMTh-His-Pro-Cc,

wherein Ww is L-tyrosine, 3-fluoro-L-tyrosine, 3-chloro-L-tyrosine, and Cc is D-alanine or L-alanine.

In some embodiments of each and every peptide provided herein, Cc is D-Alanine.

The definition of some of the abbreviations used herein are given below:

| | Chemical name of amino | |
|---|---|---|
| Abbreviation | acid or its analog | Structure of amino acid or its analog |
| Ala | L-Alanine | 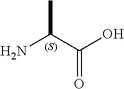 |
| D-Ala | D-Alanine | 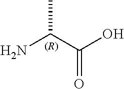 |
| NMAla | N-Methyl-L-alanine | 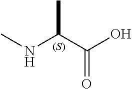 |
| NM-D-Ala | N-Methyl-D-alanine | 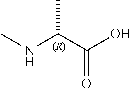 |
| Me2G | N,N-dimethylglycine | 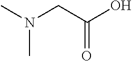 |
| Asp | L-Aspartic acid | 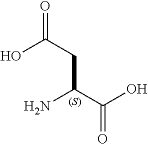 |
| D-Asp | D-Aspartic acid | 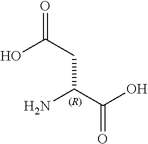 |
| Glu | L-Glutamic acid | 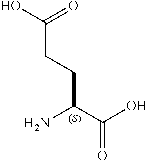 |
| D-Glu | D-Glutamic acid | 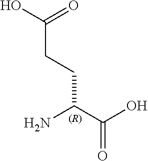 |
| N-Me-Asp | N-Methyl-L-aspartic acid | 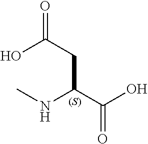 |
| N-Me-Glu | N-Methyl-L-glutamic acid | 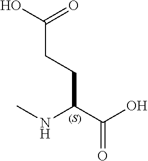 |
| Sar | Sarcosine | 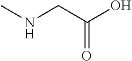 |
| MMM | Pyrrolid-1-ylacetic acid | 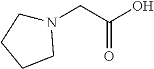 |
| NNN | Morpholin-4-ylacetic acid | 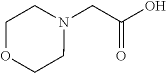 |
| Arg | L-Arginine | 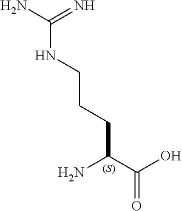 |
| Lys | L-Lysine | 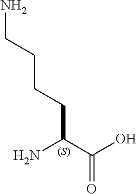 |
| Ile | L-Isoleucine | 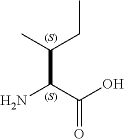 |
| Gly | Glycine | 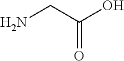 |
| Tyr | L-Tyrosine | 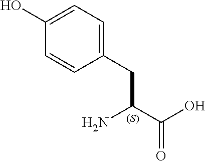 |
| OMTyr | O-Methyl-L-tyrosine | 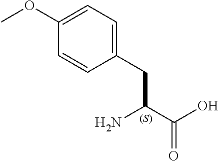 |
| Val | L-Valine | 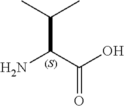 |
| Phe | L-Phenylalanine | 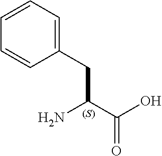 |
| D-Phe | D-Phenylalanine | 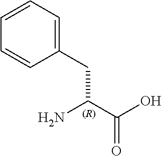 |
| AAA | 3-Hydroxy-L-tyrosine | 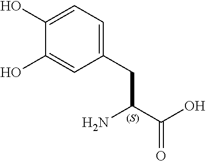 |
| BBB | 2,6-Dimethyl-L-tyrosine | 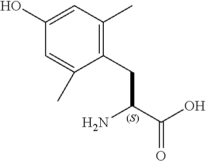 |
| CCC | 3-Fluoro-L-tyrosine | 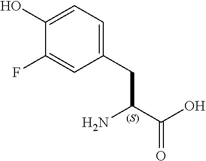 |
| DDD | 4-Fluorophenyl-L-alanine | 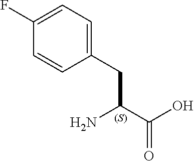 |
| EEE | 2,6-Difluoro-L-tyrosine | 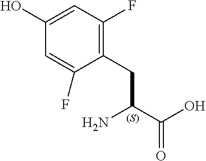 |
| FFF | 3-Nitro-L-tyrosine | 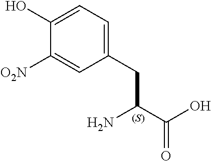 |
| GGG | 3,5-Dinitro-L-tyrosine | 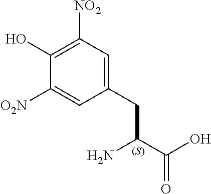 |
| HHH | 3,5-Dibromo-L-tyrosine | 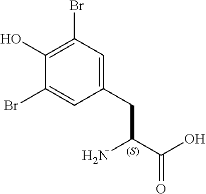 |
| III | 3-Chloro-L-tyrosine | 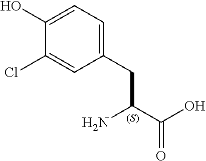 |
| JJJ | O-Allyl-L-tyrosine | 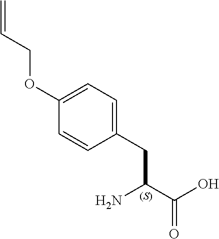 |
| OOO | 3,5-Diiodo-L-tyrosine | 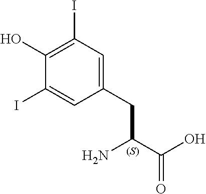 |
| His | L-Histidine | 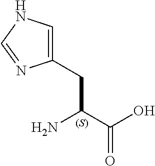 |
| OMTh | O-Methyl-L-threonine | 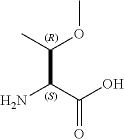 |
| NVA | L-Norvaline | 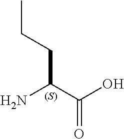 |
| Pro | L-Proline | 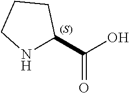 |
| D-Pro | D-Proline | 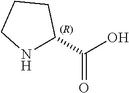 |
| | L-Histidine-amide | 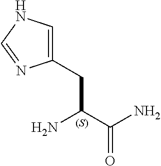 |
| | L-Proline amide | 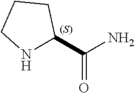 |
| | D-Proline amide | 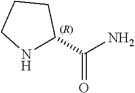 |
| | L-Isoleucine amide | 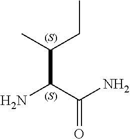 |
| | Glycine amide | 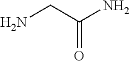 |
| | L-Alanine amide | 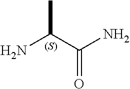 |

Cyclic forms, cyclic truncated forms, cyclic truncated dimerized forms, and cyclic truncated trimerized forms of the compounds of the above formulas may be prepared using any known method. Truncated is defined as analogs with amino acids removed from the X1 and/or X2 residues as depicted in Table 1. According to some embodiments, cyclic forms of the compounds of the above formulas may be prepared by bridging free amino and free carboxyl groups. According to some embodiments, the formation of the cyclic compounds may be conducted conventionally by treatment with a dehydrating agent by means known in the art, with suitable protection if needed. According to some embodiments, the open chain (linear form) to cyclic form reaction may involve a trans to cis isomerization of the proline. According to some embodiments, the open chain (linear form) to cyclic form reaction may involve intramolecular-cyclization.

Examples of the compounds include, but are not limited to, the compounds listed in Table 1 below.

TABLE 1

| SEQ ID | Residue (X) | | | | | | | | |
|---|---|---|---|---|---|---|---|---|---|
| NO: | X₁ | X₂ | X₃ | X₄ | X₅ | X₆ | X₇ | X₈ | |
| AngII | Asp | Arg | Val | Tyr | Ile | His | Pro | Phe | OH |
| 2 | Sar | Arg | Val | Ile | Ile | His | Pro | Ile | NH2 |
| 3 | Sar | Arg | Val | Ile | Val | His | Pro | Ile | OH |
| 5 | Sar | Arg | Val | Gly | Val | His | Pro | Gly | OH |
| 6 | Sar | Arg | Val | Gly | Val | His | Pro | Gly | NH2 |
| 7 | Sar | Arg | Val | Tyr | Val | His | Pro | Ala | OH |
| 8 | Sar | Arg | Val | Tyr | Val | His | Pro | Ala | NH2 |
| 9 | Sar | Arg | Val | Tyr | Ile | His | Pro | Ala | OH |
| 10 | Sar | Arg | Val | Tyr | Ile | His | Pro | Ala | NH2 |
| 11 | Sar | Arg | Val | Tyr | Val | His | Pro | Ile | OH |
| 12 | Sar | Arg | Val | Tyr | Val | His | Pro | Ile | NH2 |
| 13 | | Arg | Val | Tyr | Ile | His | Pro | NH2 | |
| 14 | Sar | Arg | Val | Tyr | Tyr | His | Pro | Ile | OH |
| 15 | Sar | Arg | Val | Tyr | Tyr | His | Pro | Ile | NH2 |
| 16 | Sar | Arg | Val | Tyr | Glu | His | Pro | Ile | OH |
| 19 | Sar | Arg | Val | Tyr | Tyr | His | Pro | Ala | OH |
| 20 | Sar | Arg | Val | Tyr | Glu | His | Pro | Ala | OH |
| 21 | Sar | Arg | Val | Tyr | Phe | His | Pro | Ala | OH |
| 22 | Sar | Arg | Val | Tyr | His | His | Pro | Ala | OH |
| 23 | Sar | Arg | Val | Tyr | Lys | His | Pro | Ala | OH |
| 24 | Sar | Arg | Val | Tyr | Arg | His | Pro | Ala | OH |
| 25 | Asp | Arg | Val | Tyr | Ile | His | Pro | Ala | OH |
| 26 | Sar | Arg | Val | Tyr | Tyr | His | Pro | NH2 | |
| 27 | Sar | Arg | Val | Tyr | Ile | His | Pro | D-Ala | OH |
| 28 | Sar | Arg | Val | Tyr | Tyr | His | Pro | D-Phe | OH |
| 29 | Sar | Arg | Val | OMTyr | Ile | His | Pro | Ala | OH |
| 30 | Sar | Arg | Val | Tyr | OMTh | His | Pro | Ala | OH |
| 31 | Me2G | Arg | Val | Tyr | Val | His | Pro | Ala | OH |
| 32 | Me2G | Arg | Val | Tyr | Ile | His | Pro | Ala | OH |
| 33 | NMAla | Arg | Val | Tyr | Val | His | Pro | Ala | OH |
| 34 | NMAla | Arg | Val | Tyr | Ile | His | Pro | Ala | OH |
| 35 | Sar | Arg | Val | Tyr | Ile | His | Pro | NVA | OH |
| 36 | Cyclo(Arg-Val-Tyr-Ile-His-Pro-Arg-Val-Tyr-Ile-His-Pro-Arg-Val-Tyr-Ile- | | | | | | | | |
| | His-Pro) | | | | | | | | |
| 37 | Cyclo(Phe-Val-Tyr-Ile-His-Pro-Phe-Val-Tyr-Ile-His-Pro-Phe-Val-Tyr-Ile- | | | | | | | | |
| | His-Pro) | | | | | | | | |
| 38 | Cyclo(Val-Tyr-Ile-His-Pro-Phe) | | | | | | | | |
| 40 | Sar | Arg | Val | Tyr | Glu | His | Pro | NH2 | |
| 41 | Sar | Arg | Val | Tyr | Phe | His | Pro | NH2 | |
| 42 | Sar | Arg | Val | Tyr | His | His | Pro | NH2 | |
| 43 | Sar | Arg | Val | Tyr | Lys | His | Pro | NH2 | |
| 44 | NMAla | Arg | Val | Tyr | Ile | His | Pro | D-Ala | OH |
| 45 | Sar | Arg | Val | Tyr | Arg | His | Pro | NH2 | |
| 46 | NMAla | Arg | Val | Tyr | Ile | His | Pro | D-Ala | OH |
| 47 | NM-D-Ala | Arg | Val | Tyr | Ile | His | Pro | Ala | OH |
| 48 | Sar | Arg | Val | Phe | Ile | His | Pro | D-Ala | OH |
| 49 | Sar | Lys | Val | Tyr | Ile | His | Pro | D-Ala | OH |
| 50 | Sar | Arg | Val | Tyr | Ile | Lys | Pro | D-Ala | OH |
| 51 | Sar | Arg | Val | Tyr | Ile | His | D-Pro | NH2 | |
| 52 | Sar | Arg | Val | Tyr | Tyr | His | Pro | OH | |
| 53 | Sar | Arg | Val | Tyr | D-Ala | His | Pro | NH2 | |
| 54 | Sar | Arg | Val | Tyr | Val | His | Pro | OH | |
| 55 | Sar | Arg | Val | Tyr | Val | His | NH2 | | |
| 56 | Asp | Arg | Val | Tyr | Ile | His | Pro | Gly | OH |
| 57 | Asp | Arg | Val | Tyr | Tyr | His | Pro | NH2 | |
| 58 | D-Asp | Arg | Val | Tyr | Tyr | His | Pro | NH2 | |
| 59 | Glu | Arg | Val | Tyr | Tyr | His | Pro | NH2 | |
| 60 | D-Glu | Arg | Val | Tyr | Tyr | His | Pro | NH2 | |
| 61 | N-Me-Asp | Arg | Val | Tyr | Tyr | His | Pro | NH2 | |
| 62 | N-Me-Glu | Arg | Val | Tyr | Tyr | His | Pro | NH2 | |
| 63 | Asp | Arg | Val | Tyr | Phe | His | Pro | NH2 | |
| 64 | Glu | Arg | Val | Tyr | Phe | His | Pro | NH2 | |
| 65 | N-Me-Asp | Arg | Val | Tyr | Phe | His | Pro | NH2 | |
| 66 | N-Me-Glu | Arg | Val | Tyr | Phe | His | Pro | NH2 | |
| 67 | Sar | Arg | Val | AAA | Ile | His | Pro | D-Ala | OH |
| 68 | Sar | Arg | Val | BBB | Ile | His | Pro | D-Ala | OH |
| 69 | Sar | Arg | Val | CCC | Ile | His | Pro | D-Ala | OH |
| 70 | Sar | Arg | Val | DDD | Ile | His | Pro | D-Ala | OH |
| 71 | Sar | Arg | Val | EEE | Ile | His | Pro | D-Ala | OH |
| 72 | Sar | Arg | Val | FFF | Ile | His | Pro | D-Ala | OH |
| 73 | Sar | Arg | Val | GGG | Ile | His | Pro | D-Ala | OH |
| 74 | Sar | Arg | Val | HHH | Ile | His | Pro | D-Ala | OH |
| 75 | Sar | Arg | Val | III | Ile | His | Pro | D-Ala | OH |
| 76 | Sar | Arg | Val | JJJ | Ile | His | Pro | D-Ala | OH |
| 77 | Sar | Arg | Val | AAA | OMTh | His | Pro | Ala | OH |
| 78 | Sar | Arg | Val | CCC | OMTh | His | Pro | Ala | OH |
| 79 | Sar | Arg | Val | III | OMTh | His | Pro | Ala | OH |
| 80 | MMM | Arg | Val | Tyr | Ile | His | Pro | D-Ala | OH |
| 81 | NNN | Arg | Val | Tyr | Ile | His | Pro | D-Ala | OH |
| 82 | NM-D-Ala | Arg | Val | Tyr | Ile | His | Pro | D-Ala | OH |
| 83 | Sar | Arg | Val | Tyr | NVA | His | Pro | D-Ala | OH |
| 84 | Sar | Arg | Val | Tyr | OMTh | His | Pro | D-Ala | OH |
| 85 | Sar | Arg | Val | OOO | Ile | His | Pro | D-Ala | OH |

The definition of the amino acid or its analogues, please refer to the table of abbreviation.

Accordingly, in some embodiments, a peptide is provided that comprises a sequence of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85. In some embodiments, the peptide or peptide mimetic, comprises the sequence of SEQ ID NO:27. In some embodiments, the peptide or peptide mimetic is cyclic. In some embodiments, the peptide or peptide mimetic is dimerized. In some embodiments, the peptide or peptide mimetic is trimerized.

The compounds can act as agonists of β-arrestin/GRK-mediated signal transduction via the AT1 angiotensin receptor. The ability of the compounds to affect G protein-mediated signaling may be measured using any assay known in the art used to detect G protein-mediated signaling or GPCR activity or the absence of such signaling/activity. “GPCR activity” refers to the ability of a GPCR to transduce a signal. Such activity can be measured, e.g., in a heterologous cell, by coupling a GPCR (or a chimeric GPCR) to a G-protein and a downstream effector such as PLC or adenylate cyclase, and measuring increases in intracellular calcium (see, e.g., Offermans & Simon, J. Biol. Chem. 270:15175 15180 (1995)). Receptor activity can be effectively measured by recording ligand-induced changes in [Ca²⁺]ᵢ using fluorescent Ca²⁺-indicator dyes and fluorometric imaging. A “natural ligand-induced activity” as used herein, refers to the activation of the GPCR by a natural ligand of the GPCR. Activity can be assessed using any number of endpoints to measure the GPCR activity. For example, the activity of a GPCR may be assessed using an assay such as calcium mobilization, e.g., an Aequorin luminescence assay.

Generally, assays for testing compounds that modulate GPCR-mediated signal transduction include the determination of any parameter that is indirectly or directly under the influence of a GPCR, e.g., a functional, physical, or chemical effect. It includes ligand binding, changes in ion flux, membrane potential, current flow, transcription, G-protein binding, gene amplification, expression in cancer cells, GPCR phosphorylation or dephosphorylation, signal transduction, receptor-ligand interactions, second messenger concentrations (e.g., cAMP, cGMP, IP₃, DAG, or intracellular Ca²⁺), in vitro, in vivo, and ex vivo and also includes other physiologic effects such as increases or decreases of neurotransmitter or hormone release; or increases in the synthesis of particular compounds, e.g., triglycerides. Such parameters can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole-cell currents, radioisotope efflux, inducible markers, transcriptional activation of GPCRs; ligand binding assays; voltage, membrane potential and conductance changes; ion flux assays; changes in intracellular second messengers such as cAMP and inositol triphosphate (IP₃); changes in intracellular calcium levels; neurotransmitter release, and the like.

When a G protein receptor becomes active, it binds to a G protein (e.g., Gq, Gs, Gi, Go) and stimulates the binding of GTP to the G protein. The G protein then acts as a GTPase and slowly hydrolyzes the GTP to GDP, whereby the receptor, under normal conditions, becomes deactivated. G protein-mediated signaling or GPCR activity may be measured using assay systems that are capable of detecting and/or measuring GTP binding and/or hydrolysis of GTP to GDP.

Gs stimulates the enzyme adenylyl cyclase. Gi (and Go), on the other hand, inhibit this enzyme. Adenylyl cyclase catalyzes the conversion of ATP to cAMP. Thus, constitutively activated GPCRs that couple the Gs protein are associated with increased cellular levels of cAMP. On the other hand, activated GPCRs that couple the Gi (or Go) protein are associated with decreased cellular levels of cAMP. Thus, assays that detect cAMP can be utilized to determine if a candidate compound is, e.g., an inverse agonist to the receptor (i.e., such a compound would decrease the levels of cAMP). A variety of approaches known in the art for measuring cAMP can be utilized, such as an approach that relies upon the use of anti-cAMP antibodies in an ELISA-based format. Another type of assay that can be utilized is a whole-cell second messenger reporter system assay. Promoters on genes drive the expression of the proteins that a particular gene encodes. Cyclic AMP drives gene expression by promoting the binding of a cAMP-responsive DNA binding protein or transcription factor (CREB), which then binds to the promoter at specific sites called cAMP response elements and drives the expression of the gene. Reporter systems can be constructed which have a promoter containing multiple cAMP response elements before the reporter gene, e.g., β-galactosidase or luciferase. Thus, a constitutively activated Gs-linked receptor causes the accumulation of cAMP that then activates the gene and expression of the reporter protein. The reporter protein such as β-galactosidase or luciferase can then be detected using standard biochemical assays.

Gq and Go are associated with activation of the enzyme phospholipase C, which in turn hydrolyzes the phospholipid PIP2, releasing two intracellular messengers: diacylglycerol (DAG) and inistol 1,4,5-triphoisphate (IP3). Increased accumulation of IP3 is associated with activation of Gq- and Go-associated receptors. Assays that detect IP3 accumulation can be utilized to determine if a candidate compound is, e.g., an inverse agonist to a Gq- or Go-associated receptor (i.e., such a compound would decrease the levels of IP3). Gq-dependent receptors can also be examined using an API reporter assay in that Gq-dependent phospholipase C causes activation of genes containing API elements.

Samples or assays comprising GPCRs that are treated with a potential activator, inhibitor, or modulator are compared to control samples without the inhibitor, activator, or modulator to examine the extent of inhibition. Control samples (untreated with inhibitors) are assigned a relative GPCR activity value of 100%. Inhibition of a GPCR is achieved when the GPCR activity value relative to the control is about 80%, preferably 50%, more preferably 25%. Activation of a GPCR is achieved when the GPCR activity value relative to the control (untreated with activators) is 110%, more preferably 150%, more preferably 200-500% (i.e., two to five fold higher relative to the control), more preferably 1000-3000% or higher.

The effects of the compounds upon the function of the GPCR polypeptides can be measured by examining any of the parameters described above. Any suitable physiological change that affects GPCR activity can be used to assess the influence of a compound on the GPCRs and natural ligand-mediated GPCR activity. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺, IP3 or cAMP.

For a general review of GPCR signal transduction and methods of assaying signal transduction, see, e.g., Methods in Enzymology, vols. 237 and 238 (1994) and volume 96 (1983); Bourne et al., Nature 10:349:117 27 (1991); Bourne et al., Nature 348:125 32 (1990); Pitcher et al., Annu. Rev. Biochem. 67:653 92 (1998).

Modulators of GPCR activity are tested using GPCR polypeptides as described above, either recombinant or naturally occurring. The protein can be isolated, expressed in a cell, expressed in a membrane derived from a cell, expressed in tissue or in an animal. For example, adipocytes, cells of the immune system, transformed cells, or membranes can be used to test the GPCR polypeptides described above. Modulation is tested using one of the in vitro or in vivo assays described herein. Signal transduction can also be examined in vitro with soluble or solid-state reactions, using a chimeric molecule such as an extracellular domain of a receptor covalently linked to a heterologous signal transduction domain or a heterologous extracellular domain covalently linked to the transmembrane and or cytoplasmic domain of a receptor. Furthermore, ligand-binding domains of the protein of interest can be used in vitro in soluble or solid-state reactions to assay for ligand binding.

Ligand binding to a GPCR, a domain, or chimeric protein can be tested in a number of formats. Binding can be performed in solution, in a bilayer membrane, attached to a solid phase, in a lipid monolayer, or in vesicles. Typically, in an assay, the binding of the natural ligand to its receptor is measured in the presence of a candidate modulator. Alternatively, the binding of the candidate modulator may be measured in the presence of the natural ligand. Often, competitive assays that measure the ability of a compound to compete with the binding of the natural ligand to the receptor are used. Binding can be tested by measuring, e.g., changes in spectroscopic characteristics (e.g., fluorescence, absorbance, refractive index), hydrodynamic (e.g., shape) changes, or changes in chromatographic or solubility properties.

Receptor-G-protein interactions can also be used to assay for modulators. For example, in the absence of GTP, binding of an activator such as the natural ligand will lead to the formation of a tight complex of a G protein (all three subunits) with the receptor. This complex can be detected in a variety of ways, as noted above. Such an assay can be modified to search for inhibitors. For example, the ligand can be added to the receptor and G protein in the absence of GTP to form a tight complex. Inhibitors or antagonists may be identified by looking at the dissociation of the receptor-G protein complex. In the presence of GTP, the release of the a subunit of the G protein from the other two G protein subunits serves as a criterion of activation.

An activated or inhibited G-protein will, in turn, alter the properties of downstream effectors such as proteins, enzymes, and channels. The classic examples are the activation of cGMP phosphodiesterase by transducing in the visual system, adenylate cyclase by the stimulatory G-protein, phospholipase C by Gq and other cognate G proteins, and modulation of diverse channels by Gi and other G proteins. Downstream consequences such as generation of diacyl glycerol and IP3 by phospholipase C, and in turn, for calcium mobilization, e.g., by IP3 (further discussed below) can also be examined. Thus, modulators can be evaluated for the ability to stimulate or inhibit ligand-mediated downstream effects. Candidate modulators may be assessed for the ability to inhibit calcium mobilization induced by nicotinic acid or a related compound that activates the receptor.

In other examples, the ability of a compound to inhibit GPCR activity can be determined using downstream assays such as measuring lipolysis in adipocytes, release of free fatty acids from adipose tissue, and lipoprotein lipase activity. This may be accomplished, for example, using a competition assay in which varying amounts of a compound are incubated with a GPCR.

Modulators may therefore also be identified using assays involving β-arrestin recruitment. β-arrestin serves as a regulatory protein that is distributed throughout the cytoplasm in unactivated cells. Ligand binding to an appropriate GPCR is associated with redistribution of β-arrestin from the cytoplasm to the cell surface, where it associates with the GPCR. Thus, receptor activation and the effect of candidate modulators on ligand-induced receptor activation can be assessed by monitoring β-arrestin recruitment to the cell surface. This is frequently performed by transfecting a labeled β-arrestin fusion protein (e.g., β-arrestin-green fluorescent protein (GFP)) into cells and monitoring its distribution using confocal microscopy (see, e.g., Groarke et al., J. Biol. Chem. 274(33):23263 69 (1999)).

Receptor internalization assays may also be used to assess receptor function. Upon ligand binding, the G-protein coupled receptor-ligand complex is internalized from the plasma membrane by a clathrin-coated vesicular endocytic process; internalization motifs on the receptors bind to adaptor protein complexes and mediate the recruitment of the activated receptors into clathrin-coated pits and vesicles. Because only activated receptors are internalized, it is possible to detect ligand-receptor binding by determining the amount of internalized receptor. In one assay format, cells are transiently transfected with radiolabeled receptors and incubated for an appropriate period of time to allow for ligand binding and receptor internalization. Thereafter, surface-bound radioactivity is removed by washing with an acid solution, the cells are solubilized, and the amount of internalized radioactivity is calculated as a percentage of ligand binding. See, e.g., Vrecl et al., Mol. Endocrinol. 12:1818 29 (1988) and Conway et al., J. Cell Physiol. 189(3):341 55 (2001). In addition, receptor internalization approaches have allowed real-time optical measurements of GPCR interactions with other cellular components in living cells (see, e.g., Barak et al., Mol. Pharmacol. 51(2)177 84 (1997)). Modulators may be identified by comparing receptor internalization levels in control cells and cells contacted with candidate compounds.

Another technology that can be used to evaluate GPCR-protein interactions in living cells involves bioluminescence resonance energy transfer (BRET). A detailed discussion regarding BRET can be found in Kroeger et al., J. Biol. Chem., 276(16):12736 43 (2001).

Receptor-stimulated guanosine 5′-O-(γ-Thio)-Triphosphate ([³⁵S]GTPγS) binding to G-proteins may also be used as an assay for evaluating modulators of GPCRs. [³⁵S]GTPγS is a radiolabeled GTP analog that has a high affinity for all types of G-proteins, is available with a high specific activity and, although unstable in the unbound form, is not hydrolyzed when bound to the G-protein. Thus, it is possible to quantitatively assess ligand-bound receptor by comparing stimulated versus unstimulated [³⁵S]GTPγS binding utilizing, for example, a liquid scintillation counter. Inhibitors of the receptor-ligand interactions would result in decreased [³⁵S]GTPγS binding. Descriptions of [³⁵S]GTPγS binding assays are provided in Traynor and Nahorski, Mol. Pharmacol. 47(4):848 54 (1995) and Bohn et al., Nature 408:720 23 (2000).

The ability of modulators to affect ligand-induced ion flux may also be determined. Ion flux may be assessed by determining changes in polarization (i.e., electrical potential) of the cell or membrane expressing a GPCR. One means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575 1595 (1997)). Whole cell currents are conveniently determined using the standard methodology (see, e.g., Hamil et al., PFlugers. Archiv. 391:85 (1981). Other known assays include: radiolabeled ion flux assays and fluorescence assays using voltage-sensitive dyes (see, e.g., Vestergarrd-Bogind et al., J. Membrane Biol. 88:67 75 (1988); Gonzales & Tsien, Chem. Biol. 4:269 277 (1997); Daniel et al., J. Pharmacol. Meth. 25:185 193 (1991); Holevinsky et al., J. Membrane Biology 137:59 70 (1994)). Generally, the compounds to be tested are present in the range from 1 pM to 100 mM.

In some embodiments, assays for G-protein coupled receptors include cells that are loaded with ion or voltage sensitive dyes to report receptor activity. Assays for determining the activity of such receptors can also use known agonists and antagonists for other G-protein coupled receptors and the natural ligands disclosed herein as negative or positive controls to assess the activity of tested compounds. In assays for identifying modulatory compounds (e.g., agonists, antagonists), changes in the level of ions in the cytoplasm or membrane voltage are monitored using an ion-sensitive or membrane voltage fluorescent indicator, respectively. Among the ion-sensitive indicators and voltage probes that may be employed are those disclosed in the Molecular Probes 1997 Catalog. For G-protein coupled receptors, promiscuous G-proteins such as Gα15 and Gα16 can be used in the assay of choice (Wilkie et al., Proc. Nat'l Acad. Sci. USA 88:10049 10053 (1991)). Such promiscuous G-proteins allow coupling of a wide range of receptors to signal transduction pathways in heterologous cells.

As noted above, receptor activation by ligand binding typically initiates subsequent intracellular events, e.g., increases in second messengers such as IP3, which releases intracellular stores of calcium ions. Activation of some G-protein coupled receptors stimulates the formation of inositol triphosphate (IP3) through phospholipase C-mediated hydrolysis of phosphatidylinositol (Berridge & Irvine, Nature 312:315 21 (1984)). IP3, in turn, stimulates the release of intracellular calcium ion stores. Thus, a change in cytoplasmic calcium ion levels or a change in second messenger levels such as IP3 can be used to assess G-protein coupled receptor function. Cells expressing such G-protein coupled receptors may exhibit increased cytoplasmic calcium levels as a result of contribution from both intracellular stores and via activation of ion channels, in which case it may be desirable although not necessary to conduct such assays in calcium-free buffer, optionally supplemented with a chelating agent such as EGTA, to distinguish fluorescence response resulting from calcium release from internal stores.

Other assays can involve determining the activity of receptors which, when activated by ligand binding, resulting in a change in the level of intracellular cyclic nucleotides, e.g., cAMP or cGMP, by activating or inhibiting downstream effectors such as adenylate cyclase. In one embodiment, changes in intracellular cAMP or cGMP can be measured using immunoassays. The method described in Offermanns & Simon, J. Biol. Chem. 270:15175 15180 (1995) may be used to determine the level of cAMP. Also, the method described in Felley-Bosco et al., Am. J. Resp. Cell and Mol. Biol. 11:159 164 (1994) may be used to determine the level of cGMP. Further, an assay kit for measuring cAMP and/or cGMP is described in U.S. Pat. No. 4,115,538, herein incorporated by reference.

In another embodiment, phosphatidyl inositol (PI) hydrolysis can be analyzed according to U.S. Pat. No. 5,436,128, herein incorporated by reference. Briefly, the assay involves labeling of cells with 3H-myoinositol for 48 or more hrs. The labeled cells are treated with a compound for one hour. The treated cells are lysed and extracted in chloroform-methanol-water, after which the inositol phosphates are separated by ion-exchange chromatography and quantified by scintillation counting. Fold stimulation is determined by calculating the ratio of counts per minute (cpm) in the presence of agonist to cpm in the presence of buffer control. Likewise, fold inhibition is determined by calculating the ratio of cpm in the presence of antagonist to cpm in the presence of buffer control (which may or may not contain an agonist).

In another embodiment, transcription levels can be measured to assess the effects of a test compound on ligand-induced signal transduction. A host cell containing the protein of interest is contacted with a test compound in the presence of the natural ligand for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription-based assays using reporter genes may be used as described in U.S. Pat. No. 5,436,128, herein incorporated by reference. The reporter genes can be, e.g., chloramphenicol acetyltransferase, firefly luciferase, bacterial luciferase, 0-galactosidase and alkaline phosphatase. Furthermore, the protein of interest can be used as an indirect reporter via attachment to a second reporter such as green fluorescent protein (see, e.g., Mistili & Spector, Nature Biotechnology 15:961 964 (1997)).

The amount of transcription is then compared to the amount of transcription in either the same cell in the absence of the test compound, or it may be compared with the amount of transcription in a substantially identical cell that lacks the protein of interest. A substantially identical cell may be derived from the same cells from which the recombinant cell was prepared but which had not been modified by the introduction of heterologous DNA. Any difference in the amount of transcription indicates that the test compound has in some manner altered the activity of the protein of interest.

Samples that are treated with a GPCR antagonist are compared to control samples comprising the natural ligand without the test compound to examine the extent of modulation. Control samples (untreated with activators or inhibitors) are assigned a relative GPCR activity value of 100. Inhibition of a GPCR is achieved when the GPCR activity value relative to the control is about 90%, optionally 50%, or optionally 25%. Activation of a GPCR is achieved when the GPCR activity value relative to the control is 110%, optionally 150%, 200-500%, or 1000-2000%.

### Determining β-Arrestin/GRK-Mediated Signal Transduction

The ability of the compounds to activate β-arrestin/GRK-mediated signal transduction via the AT1 angiotensin receptor may be measured using any assay known in the art used to detect β-arrestin/GRK-mediated signal transduction via the AT1 angiotensin receptor, or the absence of such signal transduction. Generally, activated GPCRs become substrates for kinases that phosphorylate the C-terminal tail of the receptor (and possibly other sites as well). Thus, an antagonist will inhibit the transfer of ³²P from gamma-labeled GTP to the receptor, which can be assayed with a scintillation counter. The phosphorylation of the C-terminal tail will promote the binding of arrestin-like proteins and will interfere with the binding of G-proteins. The kinase/arrestin pathway plays a key role in the desensitization of many GPCR receptors.

The proximal event in β-arrestin function mediated by GPCRs is recruitment to receptors following ligand binding and receptor phosphorylation by GRK's. Thus, measure of β-arrestin recruitment was used to determine ligand efficacy for β-arrestin function.

### Peptides, Derivatives and Mimetics

The terms “peptidyl” and “peptidic” as used throughout the specification and claims are intended to include active derivatives, variants, and/or mimetics of the peptides according to the present embodiments. Peptidic compounds are structurally similar bioactive equivalents of the peptides according to the present embodiments. By a “structurally similar bioactive equivalent” is meant a peptidyl compound with structure sufficiently similar to that of an identified bioactive peptide to produce substantially equivalent therapeutic effects. For example, peptidic compounds derived from the amino acid sequence of the peptide, or having an amino acid sequence backbone of the peptide, are considered structurally similar bioactive equivalents of the peptide.

The term “variant” refers to a protein or polypeptide in which one or more amino acid substitutions, deletions, and/or insertions are present as compared to the amino acid sequence of a protein or peptide and includes naturally occurring allelic variants or alternative splice variants of a protein or peptide. The term “variant” includes the replacement of one or more amino acids in a peptide sequence with a similar or homologous amino acid(s) or a dissimilar amino acid(s). In some embodiments, variants include alanine substitutions at one or more of amino acid positions. In some embodiments, substitutions include conservative substitutions that have little or no effect on the overall net charge, polarity, or hydrophobicity of the protein. Conservative substitutions are set forth in the table below. According to some embodiments, the CK polypeptides have at least 60%, 65%, 70%, 75%, 80%, 85%, 88%, 95%, 96%, 97%, 98% or 99% sequence identity with the amino acid or amino acid analogue sequences of the compound provided herein. Percent sequence identity can be determined by using software such as BLASTP using default parameters that can be found at the NCBI website.

### Conservative Amino Acid Substitutions

| | |
|---|---|
| Basic: | arginine |
| | lysine |
| | histidine |
| Acidic: | glutamic acid |
| | aspartic acid |
| Uncharged | glutamine |
| Polar: | asparagine |
| | serine |
| | threonine |
| | tyrosine |
| Non-Polar: | phenylalanine |
| | tryptophan |
| | cysteine |
| | glycine |
| | alanine |
| | valine |
| | praline |
| | methionine |
| | leucine |
| | isoleucine |

The table below sets out another scheme of amino acid substitution:

| Original | |
|---|---|
| Residue | Substitutions |
| Ala | Gly; Ser |
| Arg | Lys |
| Asn | Gln; His |
| Asp | Glu |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr; Ile |
| Phe | Met; Leu; Tyr |
| Ser | Thr |
| Thr | Ser |
| Trp | Tyr |
| Tyr | Trp; Phe |
| Val | Ile; Leu |

Other variants can consist of less conservative amino acid substitutions, such as selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to have a more significant effect on function are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine. Other variants include those designed to either generate a novel glycosylation and/or phosphorylation site(s), or those designed to delete an existing glycosylation and/or phosphorylation site(s). Variants include at least one amino acid substitution at a glycosylation site, a proteolytic cleavage site and/or a cysteine residue. Variants also include proteins and peptides with additional amino acid residues before or after the protein or peptide amino acid sequence on linker peptides. The term “variant” also encompasses polypeptides that have the amino acid sequence of the proteins/peptides provided for herein with at least one and up to 25 (e.g., 1, 2, 3, 4, 5, 6, 7, 8, 9, 10, 15, 20) additional amino acids flanking either the 3′ or 5′ end of the amino acid sequence or both.

The term “variant” also refers to a protein that is at least 60 to 99 percent identical (e.g., 60, 65, 70, 75, 80, 85, 90, 95, 98, 99, or 100%, inclusive) in its amino acid sequence of the proteins (e.g., peptides or peptide mimetics) provided herein as determined by standard methods that are commonly used to compare the similarity in position of the amino acids of two polypeptides. The degree of similarity or identity between two proteins can be readily calculated by known methods. In some embodiments, methods to determine identity are designed to give the largest match between the sequences tested. Methods to determine identity and similarity are codified in publicly available computer programs. Variants will typically have one or more (e.g., 2, 3, 4, 5, etc.) amino acid substitutions, deletions, and/or insertions as compared with the comparison protein or peptide, as the case may be.

The compounds also include compounds having one of the general formulas described herein, in addition to derivatives and/or mimetics thereof.

The term “derivative” refers to a chemically modified protein or polypeptide that has been chemically modified either by natural processes, such as processing and other post-translational modifications, but also by chemical modification techniques, as for example, by addition of one or more polyethylene glycol molecules, sugars, phosphates, and/or other such molecules, where the molecule or molecules are not naturally attached to wild-type proteins. Derivatives include salts. Such chemical modifications are well described in basic texts and in more detailed monographs, as well as in a voluminous research literature, and they are well known to those of skill in the art. It will be appreciated that the same type of modification may be present in the same or varying degree at several sites in a given protein or polypeptide. Also, a given protein or polypeptide may contain many types of modifications. Modifications can occur anywhere in a protein or polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins—Structure And Molecular Properties, 2nd Ed., T. E. Creighton, W. H. Freeman and Company, New York (1993) and Wold, F., “Posttranslational Protein Modifications: Perspectives and Prospects,” pgs. 1-12 in Posttranslational Covalent Modification Of Proteins, B. C. Johnson, Ed., Academic Press, New York (1983); Seifter et al., Meth. Enzymol. 182:626-646 (1990) and Rattan et al., “Protein Synthesis: Posttranslational Modifications and Aging,” Ann. N.Y. Acad. Sci. 663: 48-62 (1992). The term “derivatives” include chemical modifications resulting in the protein or polypeptide becoming branched or cyclic, with or without branching. Cyclic, branched and branched circular proteins or polypeptides may result from post-translational natural processes and may be made by entirely synthetic methods, as well.

According to some embodiments, the compounds may optionally include compounds wherein the N-terminus is derivatized to a —NRR¹ group; to a —NRC(O)R group; to a —NRC(O)OR group; to a —NRS(O)₂ R group; to a —NHC(O)NHR group, where R and R¹ are hydrogen or lower alkyl with the proviso that R and R¹ are not both hydrogen; to a succinimide group; to a benzyloxycarbonyl-NH—(CBZ—CH—) group; or to a benzyloxycarbonyl-NE-group having from 1 to 3 substituents on the phenyl ring selected from the group consisting of lower alkyl, lower alkoxy, chloro, and bromo.

According to some embodiments, the compounds may optionally include compounds wherein the C terminus is derivatized to —C(O)R² where R² is selected from the group consisting of lower alkoxy, and —NR³ R⁴ where R³ and R4 are independently selected from the group consisting of hydrogen and lower alkyl.

The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. Examples of peptide mimetics in this broader sense (where part of a peptide is replaced by a structure lacking peptide bonds) are described below. Whether completely or partially non-peptide, peptide mimetics according to the embodiments provide a spatial arrangement of reactive chemical moieties that closely resemble the three-dimensional arrangement of active groups in the peptide on which the peptide mimetic is based. As a result of this similar active-site geometry, the peptide mimetic has effects on biological systems that are similar to the biological activity of the peptide.

The peptides and peptide mimetics included in the compositions can be 6 to 25, 6 to 20, 6 to 15, 6 to 10, 6 to 9, 6 to 8, 7 to 25, 7 to 20, 7 to 15, 7 to 12, 7 to 10, 7 to 9, 7 to 8, 8 to 25, 8 to 20, 8 to 15, 8 to 12, 8 to 10, 8 to 9, 9 to 25, 9 to 20, 9 to 18, 9 to 15, 9 to 14, 9 to 12, 10 to 25, 10 to 20, 10 to 15, 10 to 14, 10 to 12, 12 to 25 or 12 to 20 amino acids or amino acid analogues in length.

The peptide mimetics of the embodiments are preferably substantially similar in both three-dimensional shape and biological activity to the peptides described herein. According to some embodiments, peptide mimetics can have protective groups at one or both ends of compound, and/or replacement of one or more peptide bonds with non-peptide bonds. Such modifications may render the compounds less susceptible to proteolytic cleavage than the compound itself. For instance, one or more peptide bonds can be replaced with an alternative type of covalent bond (e.g., a carbon-carbon bond or an acyl bond). Peptide mimetics can also incorporate amino-terminal or carboxyl-terminal blocking groups such as t-butyloxycarbonyl, acetyl, alkyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl, thereby rendering the mimetic less susceptible to proteolysis. Non-peptide bonds and carboxyl- or amino-terminal blocking groups can be used singly or in combination to render the mimetic less susceptible to proteolysis than the corresponding peptide/compound. Additionally, the substitution of D-amino acids for the normal L-stereoisomer can be effected, e.g., to increase the half-life of the molecule.

Thus, according to some embodiments, the compounds may optionally include a pseudopeptide bond wherein one or more of the peptidyl [—C(O)NR-] linkages (bonds) have been replaced by a non-peptidyl linkage such as —CH₂—NH—, —CH₂—S—, —CH₂—SO—, —CH₂—SO₂—, —NH—CO—, or —CH═CH— replacing a peptide bond (—CO—NH—). According to some embodiments, the compounds may optionally include a pseudopeptide bond wherein one or more of the peptidyl [—C(O)NR-] linkages (bonds) have been replaced by a non-peptidyl linkage such as a —CH₂-carbamate linkage [—CH₂—OC(O)NR—]; a phosphonate linkage; a —CH₂-sulfonamide [—CH₂—S(O)₂ NR—] linkage; a urea [—NHC(O)NH-] linkage; a —CH₂-secondary amine linkage; or an alkylated peptidyl linkage [—C(O)NR⁶— where R⁶ is lower alkyl]. In some embodiments, mimetics have from zero to all of the —C(O)NH— linkages of the replaced by a pseudopeptide.

Examples of methods of structurally modifying a peptide known in the art to create a peptide mimetic include the inversion of backbone chiral centers leading to D-amino acid residue structures that may, particularly at the N-terminus, lead to enhanced stability for proteolytical degradation without adversely affecting activity. An example is given in the paper “Tritriated D-ala¹-Peptide T Binding”, Smith C. S. et al., Drug Development Res., 15, pp. 371-379 (1988). A second method is altering cyclic structure for stability, such as N to C interchain imides and lactams (Ede et al. in Smith and Rivier (Eds.) “Peptides: Chemistry and Biology”, Escom, Leiden (1991), pp. 268-270). An example of this is given in conformationally restricted thymopentin-like compounds, such as those disclosed in U.S. Pat. No. 4,457,489 (1985), Goldstein, G. et al., the disclosure of which is incorporated by reference herein in its entirety. A third method is to substitute peptide bonds in the peptide by pseudopeptide bonds that confer resistance to proteolysis. The synthesis of peptides containing pseudopeptide bonds such as —CH₂—NH—, —CH₂—S—, —CH₂—SO—, —CH₂—SO₂—, —NH—CO— or —CH═CH— is performed either by solution methods or in a combined procedure with solid-phase synthesis using standard methods of organic chemistry. Thus, for example, the introduction of the —CH₂—NH— bond is accomplished by preparing in solution the aldehyde Fmoc-NH—CHR—CHO according to the technique described by FEHRENTZ and CASTRO (Synthesis, 676-678, 1983) and condensing it with the growing peptide chain, either on a solid phase according to the technique described by SASAKI and COY (Peptides, 8, 119-121, 1988), or in solution.

Pharmaceutical Compositions/Formulations

Pharmaceutical compositions comprising one or more of the peptides or mimetics thereof are also provided. In some embodiments, the pharmaceutical compositions can be formulated by standard techniques using one or more physiologically acceptable carriers or excipients. In some embodiments, the formulations may contain a buffer and/or a preservative. The compounds and their physiologically acceptable salts and solvates can be formulated for administration by any suitable route, including via inhalation, topically, nasally, orally, parenterally (e.g., intravenously, intraperitoneally, intravesically or intrathecally) or rectally in a vehicle comprising one or more pharmaceutically acceptable carriers, the proportion of which is determined by the solubility and chemical nature of the peptide, chosen route of administration and standard biological practice.

According to some embodiments, pharmaceutical compositions are provided comprising effective amounts of one or more compound(s) provided herein together with, for example, pharmaceutically acceptable diluents, preservatives, solubilizers, emulsifiers, adjuvants and/or other carriers. Such compositions include diluents of various buffer content (e.g., TRIS or other amines, carbonates, phosphates, amino acids, for example, glycinamide hydrochloride (especially in the physiological pH range), N-glycylglycine, sodium or potassium phosphate (dibasic, tribasic), etc. or TRIS-HCl or acetate), pH and ionic strength; additives such as detergents and solubilizing agents (e.g., surfactants such as Pluronics, Tween 20, Tween 80 (Polysorbate 80), Cremophor, polyols such as polyethylene glycol, propylene glycol, etc.), anti-oxidants (e.g., ascorbic acid, sodium metabisulfite), preservatives (e.g., Thimersol, benzyl alcohol, parabens, etc.) and bulking substances (e.g., sugars such as sucrose, lactose, mannitol, polymers such as polyvinylpyrrolidones or dextran, etc.); and/or incorporation of the material into particulate preparations of polymeric compounds such as polylactic acid, polyglycolic acid, etc. or into liposomes. Hyaluronic acid may also be used. Such compositions can be employed to influence the physical state, stability, rate of in vivo release, and rate of in vivo clearance of a compound. See, e.g., Remington's Pharmaceutical Sciences, 18th Ed. (1990, Mack Publishing Co., Easton, Pa. 18042) pages 1435-1712 which are herein incorporated by reference. The compositions can, for example, be prepared in liquid form, or can be in dried powder, such as lyophilized form. Particular methods of administering such compositions are described infra.

Where a buffer is to be included in the formulations, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, or mixtures thereof. Each one of these specific buffers constitutes an alternative embodiment. In some embodiments, the buffer is glycylglycine, sodium dihydrogen phosphate, disodium hydrogen phosphate, sodium phosphate or mixtures thereof.

Where a pharmaceutically acceptable preservative is to be included in the formulations, the preservative is selected from the group consisting of phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, or mixtures thereof. Each one of these specific preservatives constitutes an alternative embodiment although mixtures can also be provided. In some embodiments, the preservative is phenol or m-cresol.

In some embodiments, the preservative is present in a concentration from about 0.1 mg/ml to about 50 mg/ml, more preferably in a concentration from about 0.1 mg/ml to about 25 mg/ml, and most preferably in a concentration from about 0.1 mg/ml to about 10 mg/ml.

The use of a preservative in pharmaceutical compositions is well-known to the skilled person. For convenience, reference is made to Remington: The Science and Practice of Pharmacy, 19th edition, 1995.

In some embodiments, the formulation may further comprise a chelating agent where the chelating agent may be selected from salts of ethlenediaminetetraacetic acid (EDTA), citric acid, and aspartic acid, and mixtures thereof.

In some embodiments, the chelating agent is present in a concentration from 0.1 mg/ml to 5 mg/ml. In some embodiments, the chelating agent is present in a concentration from 0.1 mg/ml to 2 mg/ml. In some embodiments, the chelating agent is present in a concentration from 2 mg/ml to 5 mg/ml.

The use of a chelating agent in pharmaceutical compositions is well-known to the skilled person. For convenience, reference is made to Remington: The Science and Practice of Pharmacy, 19th edition, 1995.

In some embodiments, the formulation may further comprise a stabilizer selected from the group of high molecular weight polymers or low molecular compounds where such stabilizers include, but are not limited to, polyethylene glycol (e.g. PEG 3350), polyvinylalcohol (PVA), polyvinylpyrrolidone, carboxymethylcellulose, different salts (e.g., sodium chloride), L-glycine, L-histidine, imidazole, arginine, lysine, isoleucine, aspartic acid, tryptophan, threonine and mixtures thereof. Each one of these specific stabilizers constitutes an alternative embodiment, which can also be mixed together. In some embodiments, the stabilizer is selected from the group consisting of L-histidine, imidazole and arginine.

In some embodiments, the high molecular weight polymer is present in a concentration from 0.1 mg/ml to 50 mg/ml. In some embodiments, the high molecular weight polymer is present in a concentration from 0.1 mg/ml to 5 mg/ml. In some embodiments, the high molecular weight polymer is present in a concentration from 5 mg/ml to 10 mg/ml. In some embodiments, the high molecular weight polymer is present in a concentration from 10 mg/ml to 20 mg/ml. In some embodiments, the high molecular weight polymer is present in a concentration from 20 mg/ml to 30 mg/ml. In some embodiments, the high molecular weight polymer is present in a concentration from 30 mg/ml to 50 mg/ml.

In some embodiments, the low molecular weight compound is present in a concentration from 0.1 mg/ml to 50 mg/ml. In some embodiments, the low molecular weight compound is present in a concentration from 0.1 mg/ml to 5 mg/ml. In some embodiments, the low molecular weight compound is present in a concentration from 5 mg/ml to 10 mg/ml. In some embodiments, the low molecular weight compound is present in a concentration from 10 mg/ml to 20 mg/ml. In some embodiments, the low molecular weight compound is present in a concentration from 20 mg/ml to 30 mg/ml. In some embodiments, the low molecular weight compound is present in a concentration from 30 mg/ml to 50 mg/ml.

The use of a stabilizer in pharmaceutical compositions is well-known to the skilled person. For convenience, reference is made to Remington: The Science and Practice of Pharmacy, 19th edition, 1995.

In some embodiments, the formulation may further comprise a surfactant where a surfactant may be selected from a detergent, ethoxylated castor oil, polyglycolyzed glycerides, acetylated monoglycerides, sorbitan fatty acid esters, poloxamers, such as 188 and 407, polyoxyethylene sorbitan fatty acid esters, polyoxyethylene derivatives such as alkylated and alkoxylated derivatives (tweens, e.g. Tween-20, or Tween-80), monoglycerides or ethoxylated derivatives thereof, diglycerides or polyoxyethylene derivatives thereof, glycerol, cholic acid or derivatives thereof, lecithins, alcohols and phospholipids, glycerophospholipids (lecithins, kephalins, phosphatidyl serine), glyceroglycolipids (galactopyransoide), sphingophospholipids (sphingomyelin), and sphingoglycolipids (ceramides, gangliosides), DSS (docusate sodium, docusate calcium, docusate potassium, SDS (sodium dodecyl sulfate or sodium lauryl sulfate), dipalmitoyl phosphatidic acid, sodium caprylate, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, palmitoyl lysophosphatidyl-L-serine, lysophospholipids (e.g. 1-acyl-sn-glycero-3-phosphate esters of ethanolamine, choline, serine or threonine), alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, zwitterionic surfactants (e.g. N-alkyl-N,N-dimethylammonio-1-propanesulfonates, 3-cholamido-1-propyldimethylammonio-1-propanesulfonate, dodecylphosphocholine, myristoyl lysophosphatidylcholine, hen egg lysolecithin), cationic surfactants (quarternary ammonium bases) (e.g. cetyl-trimethylammonium bromide, cetylpyridinium chloride), non-ionic surfactants, polyethyleneoxide/polypropyleneoxide block copolymers (Pluronics/Tetronics, Triton X-100, Dodecyl β-D-glucopyranoside) or polymeric surfactants (Tween-40, Tween-80, Brij-35), fusidic acid derivatives—(e.g. sodium tauro-dihydrofusidate etc.), long-chain fatty acids and salts thereof C6-C12 (e.g. oleic acid and caprylic acid), acylcarnitines and derivatives, N_{α}-acylated derivatives of lysine, arginine or histidine, or side-chain acylated derivatives of lysine or arginine, Na-acylated derivatives of dipeptides comprising any combination of lysine, arginine or histidine and a neutral or acidic amino acid, Na-acylated derivative of a tripeptide comprising any combination of a neutral amino acid and two charged amino acids, or the surfactant may be selected from the group of imidazoline derivatives, or mixtures thereof. Each one of these specific surfactants constitutes an alternative embodiment and can also be mixed together.

The use of a surfactant in pharmaceutical compositions is well-known to the skilled person. For convenience, reference is made to Remington: The Science and Practice of Pharmacy, 19th edition, 1995.

Pharmaceutically acceptable sweeteners comprise preferably at least one intense sweetener such as saccharin, sodium or calcium saccharin, aspartame, acesulfame potassium, sodium cyclamate, alitame, a dihydrochalcone sweetener, monellin, stevioside or sucralose (4,1′,6′-trichloro-4,1′,6′-trideoxygalactosucrose), preferably saccharin, sodium or calcium saccharin, and optionally a bulk sweetener such as sorbitol, mannitol, fructose, sucrose, maltose, isomalt, glucose, hydrogenated glucose syrup, xylitol, caramel or honey.

Intense sweeteners are conveniently employed in low concentrations. For example, in the case of sodium saccharin, the concentration may range from 0.04% to 0.1% (w/v) based on the total volume of the final formulation, and preferably is about 0.06% in the low-dosage formulations and about 0.08% in the high-dosage ones. The bulk sweetener can effectively be used in larger quantities ranging from about 10% to about 35%, preferably from about 10% to 15% (w/v).

The formulations may be prepared by conventional techniques, e.g. as described in Remington's Pharmaceutical Sciences, 1985 or in Remington: The Science and Practice of Pharmacy, 19th edition, 1995, where such conventional techniques of the pharmaceutical industry involve dissolving and mixing the ingredients as appropriate to give the desired end product.

The phrase “pharmaceutically acceptable” or “therapeutically acceptable” refers to molecular entities and compositions that are physiologically tolerable and preferably do not typically produce an allergic or similar untoward reaction, such as gastric upset, dizziness and the like, when administered to a human. Preferably, as used herein, the term “pharmaceutically acceptable” means approved by a regulatory agency of the Federal or a State government or listed in the U.S.

Pharmacopeia or other generally recognized pharmacopeia (e.g., Remington's Pharmaceutical Sciences, Mack Publishing Co. (A. R. Gennaro edit. 1985)) for use in animals, and more particularly in humans.

Administration of the compounds provided herein may be carried out using any method known in the art. For example, administration may be transdermal, parenteral, intravenous, intra-arterial, subcutaneous, intramuscular, intracranial, intraorbital, ophthalmic, intraventricular, intracapsular, intraspinal, intracisternal, intraperitoneal, intracerebroventricular, intrathecal, intranasal, aerosol, by suppositories, or oral administration. In some embodiments, a pharmaceutical composition can be for administration for injection, or for oral, pulmonary, nasal, transdermal, ocular administration. In some embodiments, the formulation is suitable for inhalation, such as either through the nose or mouth.

For oral administration, the peptide or a therapeutically acceptable salt thereof can be formulated in unit dosage forms such as capsules or tablets. The tablets or capsules may be prepared by conventional means with pharmaceutically acceptable excipients, including binding agents, for example, pregelatinized maize starch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulphate. Tablets can be coated by methods well known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

For topical administration, the peptide can be formulated in a pharmaceutically acceptable vehicle containing 0.1 to 10 percent, preferably 0.5 to 5 percent, of the active compound(s). Such formulations can be in the form of a cream, lotion, sublingual tablet, aerosols and/or emulsions and can be included in a transdermal or buccal patch of the matrix or reservoir type as are conventional in the art for this purpose.

For parenteral administration, the compounds can be administered by either intravenous, subcutaneous, or intramuscular injection in compositions with pharmaceutically acceptable vehicles or carriers. The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles and can contain formulatory agents, for example, suspending, stabilizing, and/or dispersing agents. Alternatively, the active ingredient can be in powder form for constitution with a suitable vehicle, for example, sterile pyrogen-free water, before use.

For administration by injection, in some embodiments, compound(s) in solution in a sterile aqueous vehicle which may also contain other solutes such as buffers or preservatives as well as sufficient quantities of pharmaceutically acceptable salts or of glucose to make the solution isotonic are used. In some embodiments, the pharmaceutical compositions may be formulated with a pharmaceutically acceptable carrier to provide sterile solutions or suspensions for injectable administration. In particular, injectables can be prepared in conventional forms, either as liquid solutions or suspensions, solid forms suitable for solution or suspensions in liquid prior to injection or as emulsions. Suitable excipients are, for example, water, saline, dextrose, mannitol, lactose, lecithin, albumin, sodium glutamate, cysteine hydrochloride, or the like. In addition, if desired, the injectable pharmaceutical compositions may contain minor amounts of nontoxic auxiliary substances, such as wetting agents, pH buffering agents, and the like. If desired, absorption enhancing preparations (e.g., liposomes) may be utilized. Suitable pharmaceutical carriers are described in “Remington's Pharmaceutical Sciences” by E. W. Martin.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch. For intranasal administration, the composition or compounds may be used, for example, as a liquid spray, as a powder or in the form of drops.

The compounds can also be formulated in rectal compositions, for example, suppositories or retention enemas, for example, containing conventional suppository bases, for example, cocoa butter or other glycerides.

Furthermore, the compounds can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

### Dosages

The compounds may be administered to a patient at therapeutically effective doses to prevent, treat, or control diseases and disorders mediated, in whole or in part, by a GPCR-ligand interaction, such as ARDS. Pharmaceutical compositions comprising one or more of compounds may be administered to a patient in an amount sufficient to elicit an effective protective or therapeutic response in the patient. An amount adequate to accomplish this is defined as a “therapeutically effective dose.” The dose will be determined by the efficacy of the particular compound employed and the condition of the subject, as well as the bodyweight or surface area of the area to be treated. The size of the dose also will be determined by the existence, nature, and extent of any adverse effects that accompany the administration of a particular compound or vector in a particular subject.

Toxicity and therapeutic efficacy of such compounds can be determined by standard pharmaceutical procedures in cell cultures or experimental animals, for example, by determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Compounds that exhibit large therapeutic indices can be chosen. While compounds that exhibit toxic side effects can be used, care should be taken to design a delivery system that targets such compounds to the site of affected tissue to minimize potential damage to normal cells and, thereby, reduce side effects.

The data obtained from cell culture assays and animal studies can be used to formulate a dosage range for use in humans. The dosage of such compounds lies preferably within a range of circulating concentrations that include the ED50 with little or no toxicity. The dosage can vary within this range depending upon the dosage form employed and the route of administration. For any compound used in the methods provided herein, a therapeutically effective dose can be estimated initially from cell culture assays. A dose can be formulated in animal models to achieve a circulating plasma concentration range that includes the IC50 (the concentration of the test compound that achieves a half-maximal inhibition of symptoms) as determined in cell culture. Such information can be used to more accurately determine useful doses in humans. Levels in plasma can be measured, for example, by high-performance liquid chromatography (HPLC). In general, the dose equivalent of a modulator is from about 1 ng/kg to 10 mg/kg for a typical subject.

The amount and frequency of administration of the compounds and/or the pharmaceutically acceptable salts thereof will be regulated according to the judgment of the attending clinician considering such factors as age, condition and size of the patient as well as the severity of the symptoms being treated. An ordinarily skilled physician or veterinarian can readily determine and prescribe the effective amount of the drug required to prevent, counter or arrest the progress of the condition. In general, it is contemplated that an effective amount would be from 0.001 mg/kg to 10 mg/kg body weight, and in particular from 0.01 mg/kg to 1 mg/kg body weight. More specifically, it is contemplated that an effective amount would be to continuously infuse by intravenous administration from 0.01 micrograms/kg body weight/min to 100 micrograms/kg body weight/min for a period of 12 hours to 14 days. It may be appropriate to administer the required dose as two, three, four or more sub-doses at appropriate intervals throughout the day. Said sub-doses may be formulated as unit dosage forms, for example, containing 0.01 to 500 mg, and in particular 0.1 mg to 200 mg of active ingredient per unit dosage form.

In some embodiments, the pharmaceutical preparation is in a unit dosage form. In such form, the preparation is subdivided into suitably sized unit doses containing appropriate quantities of the active component, e.g., an effective amount to achieve the desired purpose. The quantity of active compound in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg, according to the particular application. The actual dosage employed may be varied depending upon the requirements of the patient and the severity of the condition being treated. Determination of the proper dosage regimen for a particular situation is within the skill of the art. For convenience, the total dosage may be divided and administered in portions during the day as required.

In some embodiments, the peptide is administered to the subject at a rate of about 1 mg/hr to about 20 mg/hr, about 5 mg/hr to about 20 mg/hr, about 10 mg/hr to about 20 mg/hr, about 10 mg/hr to about 15 mg/hr, about 8 mg/hr to about 15 mg/hr, about 9 mg/hr to about 15 mg/hr, about 12 mg/hr to about 13 mg/hr, about 1 mg/hr, about 2 mg/hr, about 3 mg/hr, about 4 mg/hr, about 5 mg/hr, about 6 mg/hr, about 7 mg/hr, about 8 mg/hr, about 9 mg/hr, about 10 mg/hr, about 11 mg/hr, about 12 mg/hr, about 13 mg/hr, about 14 mg/hr, about 15 mg/hr, about 16 mg/hr, about 17 mg/hr, about 18 mg/hr, about 19, mg/hr, or about 20 mg/hr. In some embodiments, the peptide is administered at a rate of about 12 mg/hour. In some embodiments, the peptide is administered for up to 1, 2, 3, 4, 5, 6, 7, 8, 9, 10, 11, 12, 13, 14, 15, 16, 17, 18, 19, 20, 21, 22, 23, or 24 days. In some embodiments, the peptide is administered for up to 7 days.

### Medical Use

The compounds and compositions provided herein can be used to treating ARDS as provided for herein. In some embodiments, intravenous injection or infusion is utilized for such conditions, but the compounds can also be formulated for inhalation. Such a method would comprise administering a therapeutically effective amount of one or more compounds to a subject in need thereof. In some embodiments, the ARDS is viral induced ARDS. In some embodiments, the ARDS is induced by an influenza virus or a coronavirus. In some embodiments, the ARDS is caused by COVID-19 infection. In some embodiments, the ARDS is in a subject who has a viral infection. In some embodiments, the viral infection is an influenza viral infection. In some embodiments, the viral infection is a coronavirus infection. In some embodiments, the coronavirus infection is a COVID-19 infection. In some embodiments, the subject having the infection is a subject in need thereof.

In some embodiments, the compounds and compositions provided herein can be used to prevent or treat thrombosis as provided for herein. In some embodiments, intravenous injection or infusion is utilized for such conditions, but the compounds can also be formulated for inhalation. Such a method can comprise administering a therapeutically effective amount of one or more compounds as provided herein to a subject in need thereof. In some embodiments, the thrombosis is the formation of a thrombus or a blood clot. In some embodiments, the thrombosis is the formation of a thrombus. In some embodiments, the thrombosis is the formation of a blood clot. In some embodiments, the thrombosis is venous thrombosis. In some embodiments, the thrombosis is deep vein thrombosis. In some embodiments, the thrombosis is Paget-Schroetter disease. In some embodiments, the thrombosis is Budd-Chiari syndrome, portal vein thrombosis. In some embodiments, the thrombosis is renal vein thrombosis. In some embodiments, the thrombosis is cerebral venous sinus thrombosis. In some embodiments, the thrombosis is jugular vein thrombosis. In some embodiments, the thrombosis is cavernous sinus thrombosis. In some embodiments, the thrombosis is arterial thrombosis. In some embodiments, the thrombosis is stroke. In some embodiments, the thrombosis is myocardial infarction. In some embodiments, the thrombosis is limb ischemia. In some embodiments, the subject having the thrombosis is a subject in need thereof. In some embodiments, the thrombosis is caused by a viral infection. In some embodiments, the thrombosis is caused by a coronavirus infection. In some embodiments, the coronavirus is SARS, MERS, or COVID-19.

In some embodiments, the compounds and compositions provided herein can be used to prevent or reduce platelet adhesion and/or platelet aggregation in a subject in need thereof as provided for herein. In some embodiments, intravenous injection or infusion is utilized for such conditions, but the compounds can also be formulated for inhalation. Such a method can comprise administering a therapeutically effective amount of one or more compounds as provided herein to a subject in need thereof. In some embodiments, the platelet adhesion in the subject is reduced. In some embodiments, the platelet adhesion is the platelet adhesion over the collagen matrix. In some embodiments, the platelet aggregation in the subject is reduced. In some embodiments, the subject is at risk of developing thrombosis. In some embodiments, the thrombosis or the risk of developing thrombosis is caused by a viral infection. In some embodiments, the thrombosis is caused by a coronavirus infection. In some embodiments, the coronavirus is SARS, MERS, or COVID-19.

Additionally, it has been found that D-Dimer formation and response can be used as an indirect measure of thrombosis. (Vidali, et al., ERJ Open Research 2020 6: 00260-2020). Compounds that can reduce IAS and FAS D-Dimer response by greater than 30% can also be used as an indirect method of showing efficacy. Accordingly, in some embodiments, the present compounds, peptides, compositions, and pharmaceutical compositions provided for herein can be used to treat or reduce a D-Dimer response. In some embodiments, methods of reducing a D-Dimer response in a subject with ARDS or a viral infection are provided. In some embodiments, the method comprises administering to the subject a peptide or peptide mimetic as provided for herein. In some embodiments, intravenous injection or infusion is utilized for such conditions, but the compounds can also be formulated for inhalation. In some embodiments, the D-Dimer response of the subject is reduced compared to a subject with the ARDS or the viral infection but without being administered the peptide or peptide mimetic. In some embodiments, the D-Dimer response or formation is reduced by greater than 10, 20, 30, 40, 50, 60, 70, 80, 90, 95, or 99%. In some embodiments, the D-Dimer response is an IAS D-Dimer response. In some embodiments, the D-Dimer response is a FAS D-Dimer response. In some embodiments, the subject is at risk for developing thrombosis or has developed thrombosis. In some embodiments, the viral infection is a coronavirus infection. In some embodiments, the coronavirus is SARS, MERS, or COVID-19. In some embodiments, the coronavirus is COVID-19. In some embodiments, the peptide has a sequence of SEQ ID NO: 27. In some embodiments, the reduction of D-Dimer response is statistically significant. In some embodiments, the reduction of D-Dimer response is clinically significant. In some embodiments, the reduction of D-Dimer response is clinically important. In some embodiments, the reduction of D-Dimer response is clinically meaningful.

In some embodiments, methods of reducing a D-Dimer level in circulation in a subject with ARDS or a viral infection are also provided. In some embodiments, the method comprises administering to the subject a peptide or peptide mimetic as provided for herein. In some embodiments, intravenous injection or infusion is utilized for such conditions, but the compounds can also be formulated for inhalation. In some embodiments, the D-Dimer level of the subject is reduced compared to a subject with the ARDS or the viral infection but without being administered the peptide or peptide mimetic. In some embodiments, the D-Dimer level is reduced by greater than 10, 20, 30, 40, 50, 60, 70, 80, 90, 95, or 99%. In some embodiments, the D-Dimer level is an IAS D-Dimer level. In some embodiments, the D-Dimer response is a FAS D-Dimer level. In some embodiments, the subject is at risk for developing thrombosis or has developed thrombosis. In some embodiments, the viral infection is a coronavirus infection. In some embodiments, the coronavirus is SARS, MERS, or COVID-19. In some embodiments, the coronavirus is COVID-19. In some embodiments, the peptide has a sequence of SEQ ID NO: 27. In some embodiments, the reduction of D-Dimer level is statistically significant. In some embodiments, the reduction of D-Dimer level is clinically significant. In some embodiments, the reduction of D-Dimer level is clinically important. In some embodiments, the reduction of D-Dimer level is clinically meaningful.

In some embodiments, methods of reducing a length of hospital stay of a subject with ARDS or a viral infection are provided. In some embodiments, methods of reducing a length of hospital stay of a subject with ARDS are provided. In some embodiments, methods of reducing a length of hospital stay of a subject with a viral infection are provided. In some embodiments, the method comprises administering to the subject a peptide or peptide mimetic as provided for herein. In some embodiments, intravenous injection or infusion is utilized for such conditions, but the compounds can also be formulated for inhalation. In some embodiments, the length of hospital stay of the subject is reduced compared to a subject with the ARDS or the viral infection but without being administered the peptide or peptide mimetic. In some embodiments, the viral infection is a coronavirus infection. In some embodiments, the coronavirus is SARS, MERS, or COVID-19. In some embodiments, the coronavirus is COVID-19. In some embodiments, the length of hospital stay of the subject is reduce compared to a subject with the ARDS or the viral infection but without being administered the peptide. In some embodiments, the length of hospital stay is reduced by about 1, 2, 3, 4, 5, 6, 7, 8, 9, 10, 11, 12, 13, 14, 15, 16, 17, 18, 19, 20, 21, 22, 23, or 24 days. In some embodiments, the length of hospital stay is reduced by about 4 days. In some embodiments, the length of hospital stay is reduced by about 12 days. In some embodiments, the peptide has a sequence of SEQ ID NO: 27. In some embodiments, the peptide is administered to the subject at a rate of about 1 mg/hr to about 20 mg/hr, about 5 mg/hr to about 20 mg/hr, about 10 mg/hr to about 20 mg/hr, about 10 mg/hr to about 15 mg/hr, about 8 mg/hr to about 15 mg/hr, about 9 mg/hr to about 15 mg/hr, about 12 mg/hr to about 13 mg/hr, about 1 mg/hr, about 2 mg/hr, about 3 mg/hr, about 4 mg/hr, about 5 mg/hr, about 6 mg/hr, about 7 mg/hr, about 8 mg/hr, about 9 mg/hr, about 10 mg/hr, about 11 mg/hr, about 12 mg/hr, about 13 mg/hr, about 14 mg/hr, about 15 mg/hr, about 16 mg/hr, about 17 mg/hr, about 18 mg/hr, about 19, mg/hr, or about 20 mg/hr. In some embodiments, the reduction of the length of hospital stay is statistically significant. In some embodiments, the reduction of the length of hospital stay is clinically significant. In some embodiments, the reduction of the length of hospital stay is clinically important. In some embodiments, the reduction of the length of hospital stay is clinically meaningful.

### Definitions

Unless otherwise defined, all technical and scientific terms used herein have the same meaning as commonly understood by one of ordinary skill in the art. Although methods and materials similar or equivalent to those described herein can be used in the practice or testing, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages will be apparent from the following detailed description and claims.

For the purposes of promoting an understanding of the embodiments described herein, reference will be made to some embodiments and specific language will be used to describe the same. The terminology used herein is for the purpose of describing particular embodiments only, and is not intended to limit the scope of the embodiments. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. Thus, for example, a reference to “a host cell” includes a plurality of such host cells, and a reference to “an antibody” is a reference to one or more antibodies and equivalents thereof known to those skilled in the art, and so forth.

As used herein, the terms “comprise,” “have,” “has,” and “include” and their conjugates, as used herein, mean “including but not limited to.” While various compositions and methods are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

### Examples

The following examples are illustrative, but not limiting, of the methods and compositions. Other suitable modifications and adaptations of the variety of conditions and parameters normally encountered in therapy and that are obvious to those skilled in the art are within the spirit and scope of the embodiments.

### Example 1: Synthesis of Compounds

Peptides and intermediates described herein were prepared by the solid-phase method of peptide synthesis. (cf. R. Merrifield J. Am. Chem. Soc. 1964, 85, 2149; M. Bodansky, “Principles of Peptide Synthesis.” Springer-Verlag, 1984.) The peptide synthesis and purification procedures employed were standard methods well described in the art, including, but not limited to, amino acid coupling procedures, wash steps, deprotection procedures, resin cleavage procedures, and ion exchange and HPLC purification methods using commercial automated peptide synthesizers and commercially available resins and protected amino acids. More specifically, the peptides were synthesized from their C-terminus by stepwise addition of Fmoc-protected amino acids (pre-activated or in situ activated) and deprotection of the Fmoc group with piperidine to an acid-labile linker attached to an insoluble support resin. Following synthesis, the resin-bound peptide was side chain-deprotected and detached from the resin with trifluoroacetic acid and cation scavengers. Peptides were purified by aqueous extraction or by precipitation from organic solvents such as ether or t-butyl methyl ether followed by centrifugation and decanting and/or by HPLC and lyophilization. The peptides can also be synthesized according to methods provided in U.S. Pat. No. 9,611,293, which is hereby incorporated by reference in its entirety. The peptides can also be crystallized as provided in U.S. Pat. No. 9,518,086, which is hereby incorporated by reference in its entirety

### Example 2: β-Arrestin Recruitment Assay

The proximal event in β-arrestin function mediated by GPCRs is recruitment to receptors following ligand binding and receptor phosphorylation by GRK's. Thus, the measure of β-arrestin recruitment was used to determine ligand efficacy for β-arrestin function.

B-arrestin-2 recruitment to the human and rat angiotensin 2 type 1 receptor (human AT1R and rat AT1aR, respectively) was measured with the PathHunter™ β-arrestin assay (DiscoveRx Corporation, Fremont CA). Cells, plasmid(s), and detection reagent(s) were purchased from DiscoveRx, and assays were performed per the manufacturer's instructions. Human AT1R and rat AT1aR were cloned into the pCMV-ProLink vector, verified by sequencing, and transfected into PathHunter β-arrestin HEK293 cells. Stably transfected clonal cell lines were selected with Hygromycin and G418. These clonal cell lines were used for all experiments.

For assays, 25,000 cells were seeded per well in 96-well microplates in volumes of 90 μL and allowed to grow overnight at 37° C. in 5% CO₂. Peptides provided herein were dissolved in deionized water to a concentration of 1 mM. Peptides were then further diluted in assay buffer (Hank's balanced salt solution with 20 mM HEPES) to add peptide to the cells to reach final concentrations ranging from 10 μM to 1 pM. Cells were then incubated for 60 minutes at 37° C. in 5% CO₂, followed by the addition of 50 μL of PathHunter Detection Reagent to each well.

The microplates were then incubated at room temperature for 60 minutes, and then luminescence was measured using a NOVOstar microplate reader purchased from BMG Labtech. B-arrestin-2 recruitment to receptors was measured as relative luminescence intensity expressed in arbitrary units. Results are displayed in Table 2 below.

### Example 3: IP1 Accumulation Assay

A secondary measure of G protein coupling efficacy was also performed. IP3 is generated by activation of phospholipase C by Gα-q. IP3 is degraded to IP1, which can be forced to accumulate in cells by blocking degradation with lithium chloride. Thus, we measured the accumulation of IP1 to determine ligand efficacy for G protein activation.

IP1 accumulation generated by human and rat angiotensin 2 type 1 receptor (human AT1R and rat AT1aR, respectively) was measured with IP-One Tb kits purchased from Cisbio and used per the manufacturer's instructions. Clonal stably transfected cell lines expressing human AT1TR or rat AT1aR were used for all experiments.

For assays, 4,000 cells were seeded per well in 384-well small-volume microplates in volumes of 20 uL and allowed to grow overnight at 37° C. in 5% CO₂. Cell growth media was then replaced with stimulation buffer supplied by Cisbio containing 50 mM lithium chloride. Peptides TRV-120,001 through TRV-120,035 were dissolved in deionized water to a concentration of 1 mM. For agonist detection, peptides were then further diluted in stimulation buffer to add peptide to the cells to reach final concentrations ranging from 10 uM to 1 pM. For antagonist detection, peptides were then further diluted in stimulation buffer to add peptide to the cells to reach final concentrations ranging from 10 uM to 1 pM, and followed by the addition of 10 nM AngII. Following the addition of peptides, cells were incubated at 37° C. in 5% CO₂ for 30 minutes and then lysed with detection reagents added per manufacturer's instructions. Microplates were incubated for 60-90 minutes and then time-resolved fluorescence intensities were measured using a PHERAstar Plus microplate reader from BMG Labtech. IP1 accumulation was measured as a change in ratio of time-resolved fluorescent intensities measured at 665 nm and 620 nm. Results are displayed in Table 2 below.

TABLE 2
Biological activity.

| | human AT1R | | | | rat AT1aR | | | |
|---|---|---|---|---|---|---|---|---|
| | b-arrestin2 | | IP One | | b-arrestin2 | | IP One | |
| | EC50 | Max | EC50 | Max | EC50 | Max | EC50 | Max |
| # | (M) | (%) | (M) | (%) | (M) | (%) | (M) | (%) |
| AngII | 9.7E−09 | 99.0 | 2.6E−09 | 96.9 | 6.8E−09 | 97.0 | 1.3E−09 | 98.1 |
| Valsartan | inactive | | inactive | | inactive | | | |
| 2 | 4.5E−06 | 24.9 | inactive | | inactive | | 3.6E−07 | 33.8 |
| 3 | 4.5E−07 | 36.5 | inactive | | 1.2E−07 | 67.1 | 4.1E−08 | 19.6 |
| 5 | 1.6E−06 | 39.4 | 6.5E−08 | 18.1 | 1.9E−06 | 61.7 | 9.6E−08 | 24.8 |
| 6 | 9.2E−07 | 12.1 | 1.1E−07 | 14.1 | inactive | | 5.9E−08 | 21.9 |
| 7 | 1.1E−08 | 63.0 | inactive | | 7.5E−09 | 86.5 | 6.0E−09 | 9.3 |
| 8 | 2.1E−07 | 36.9 | inactive | | 2.0E−07 | 41.8 | 3.5E−08 | 11.3 |
| 9 | 9.2E−09 | 69.2 | inactive | | 4.8E−09 | 74.0 | 6.6E−09 | 9.5 |
| 10 | 1.6E−07 | 55.2 | inactive | | 1.8E−07 | 60.3 | 2.2E−07 | 13.3 |
| 11 | 3.4E−09 | 58.9 | inactive | | 6.1E−09 | 80.7 | 3.5E−09 | 24.4 |
| 12 | 3.9E−07 | 19.9 | 1.9E−07 | 20.7 | 1.2E−07 | 34.4 | 7.9E−08 | 44.9 |
| 13 | 4.6E−06 | 43.4 | inactive | | 4.3E−05 | 165.3 | Inactive | |
| 14 | 8.0E−09 | 59.3 | 1.6E−07 | 13.5 | 1.4E−08 | 79.0 | 3.2E−08 | 19.3 |
| 15 | 8.2E−07 | 44.3 | 2.5E−07 | 19.3 | 2.3E−07 | 62.1 | 7.7E−08 | 51.1 |
| 16 | 8.8E−07 | 56.9 | inactive | | 1.4E−07 | 76.9 | 9.2E−08 | 24.4 |
| 19 | 3.1E−08 | 67.9 | inactive | | 1.8E−08 | 91.2 | Inactive | |
| 20 | inactive | | inactive | | 3.6E−06 | 86.6 | Inactive | |
| 21 | 3.3E−08 | 64.4 | inactive | | 1.3E−08 | 82.7 | Inactive | |
| 22 | 2.7E−07 | 54.5 | inactive | | 2.7E−08 | 74.3 | 2.5E−07 | 5.2 |
| 23 | 4.4E−08 | 61.2 | inactive | | 3.9E−08 | 84.9 | 1.1E−08 | 10.3 |
| 24 | 4.8E−07 | 61.3 | inactive | | 6.3E−08 | 74.1 | 7.4E−09 | 15.1 |
| 25 | 2.1E−08 | 57.9 | inactive | | 1.5E−08 | 77.0 | 7.3E−09 | 14.1 |
| 26 | 7.5E−08 | 51.9 | inactive | | 3.8E−08 | 77.3 | Inactive | |
| 27 | 1.7E−08 | 58.1 | inactive | | 1.6E−08 | 84.2 | Inactive | |
| 28 | 2.0E−08 | 82.9 | inactive | | 1.9E−08 | 91.4 | 6.4E−08 | 24.6 |
| 29 | 5.9E−07 | 43.3 | inactive | | 2.0E−07 | 64.8 | Inactive | |
| 30 | 9.2E−09 | 68.2 | inactive | | 8.2E−09 | 80.4 | Inactive | |
| 31 | 1.0E−07 | 58.1 | inactive | | 3.7E−08 | 75.9 | 1.3E−08 | 6.2 |
| 32 | 7.9E−08 | 55.6 | inactive | | 4.3E−08 | 80.2 | 2.4E−08 | 6.8 |
| 33 | 4.0E−08 | 54.9 | inactive | | 3.1E−08 | 86.9 | Inactive | |
| 34 | 2.5E−08 | 53.0 | inactive | | 3.2E−08 | 81.7 | Inactive | |
| 35 | 1.2E−08 | 67.9 | 2.7E−09 | 7.1 | 9.3E−09 | 79.8 | 1.2E−08 | 30.5 |
| 36 | 1.2E−06 | 39.5 | inactive | | 1.9E−06 | 32.8 | 1.0E−07 | 19.2 |
| 37 | 1.7E−06 | 62.3 | inactive | | 1.9E−06 | 60.7 | 2.4E−07 | 14.7 |
| 38 | 6.8E−07 | 43.1 | inactive | | 2.5E−06 | 66.4 | 9.2E−07 | 20.8 |
| 44 | 8.0E−08 | 47.3 | inactive | | 6.5E−08 | 71.9 | 8.2E−09 | 14.6 |
| 57 | 4.3E−07 | 42.06 | 3.6E−09 | 10.03 | 4.5E−07 | 64.38 | 3.3E−07 | 35.26 |
| 58 | 3.3E−07 | 48.67 | inactive | | 2.6E−07 | 81.1 | 1.4E−06 | 9.937 |
| 59 | 1.4E−06 | 19.49 | inactive | | 8.2E−07 | 27.84 | Inactive | |
| 60 | 3.4E−07 | 44.02 | inactive | | 1.8E−07 | 79.27 | Inactive | |
| 61 | 1.2E−06 | 37.55 | inactive | | 2.1E−06 | 60.39 | Inactive | |
| 62 | 1.3E−06 | 24.68 | inactive | | 3.9E−06 | 44.19 | Inactive | |
| 63 | 5.3E−07 | 35.93 | inactive | | 5.3E−07 | 50.13 | Inactive | |
| 64 | 1.2E−06 | 20.39 | inactive | | 8.7E−07 | 26.95 | Inactive | |
| 65 | 2.0E−06 | 37.8 | inactive | | 2.8E−06 | 67.27 | Inactive | |
| 66 | 6.7E−06 | 30.56 | inactive | | 1.0E−05 | 58.54 | Inactive | |
| 67 | 1.0E−07 | 100.6 | inactive | | 6.3E−08 | 117 | Inactive | |
| 68 | 6.3E−07 | 39.2 | inactive | | 4.0E−07 | 59.2 | 2.5E−05 | 21.2 |
| 69 | 1.3E−08 | 53.9 | inactive | | 1.0E−08 | 88.3 | Inactive | |
| 70 | 1.6E−07 | 29.1 | inactive | | 7.9E−08 | 55.6 | Inactive | |
| 71 | 4.0E−07 | 52.7 | inactive | | 1.6E−07 | 81.7 | Inactive | |
| 72 | 6.3E−08 | 62.6 | inactive | | 2.5E−08 | 88.6 | Inactive | |
| 73 | 5.0E−06 | 46.8 | inactive | | 4.0E−06 | 77.4 | 2.5E−06 | 20.8 |
| 74 | 2.5E−07 | 47.8 | inactive | | 1.0E−07 | 59.0 | Inactive | |
| 75 | 2.5E−08 | 55.0 | inactive | | 1.3E−08 | 79.3 | 2.5E−05 | 26.7 |
| 76 | 1.6E−06 | 43.0 | inactive | | 6.3E−07 | 55.5 | Inactive | |
| 77 | 5.0E−08 | 97.5 | inactive | | 4.0E−08 | 110.5 | Inactive | |
| 78 | 1.0E−08 | 71.5 | inactive | | 5.0E−09 | 91.3 | Inactive | |
| 79 | 7.9E−09 | 64.8 | inactive | | 7.9E−09 | 93.6 | Inactive | |
| 80 | 1.0E−06 | 58.5 | inactive | | 5.0E−07 | 82.9 | Inactive | |
| 81 | 1.0E−05 | 32.6 | inactive | | 1.0E−05 | 54.3 | Inactive | |
| 82 | 3.2E−07 | 38.3 | inactive | | 1.3E−07 | 60.6 | Inactive | |
| 83 | 2.0E−07 | 34.3 | inactive | | 5.0E−08 | 70.2 | Inactive | |
| 84 | 3.2E−08 | 50.5 | inactive | | 1.6E−08 | 80.6 | Inactive | |
| 85 | 1.6E−06 | 51.1 | | | 5.0E−07 | 75.5 | 1.6E−05 | 23.4 |

Note:
inactive means that EC50 was greater than 10 uM..

### Example 4: Calcium Mobilization Assay

G protein efficacy can be measured in many ways. GPCRs that couple to the G_{q} subclass of heterotrimeric g proteins activate a wide array of signal transduction when activated by agonists. One of the most commonly measured pathways is the activation of phospholipase C by Galpha-q, which cleaves phosphatidylinositol bisphosphate to release IP3; IP3, in turn, releases calcium to the cytosol from intracellular stores via the IP3 receptor. Thus, we measured intracellular free calcium to determine ligand efficacy for G protein activation.

Intracellular free calcium generated by human and rat angiotensin 2 type 1 receptor (human AT1R and rat AT1aR, respectively) was measured with Fluo-4 NW kits purchased from Invitrogen and used per the manufacturer's instructions. Clonal stably transfected cell lines expressing human AT1TR or rat AT1aR were used for all experiments.

For assays, 25,000 cells were seeded per well in 96-well microplates in volumes of 90 uL and allowed to grow overnight at 37° C. in 5% CO₂. Fluo-4 NW dye was mixed with probenecid and assay buffer (Hank's balanced salt solution with 20 mM HEPES), and cell growth media was replaced with this mixture, followed by incubation for 30-45 minutes at 37° C. in 5% CO₂. Peptides TRV-120,001 through TRV-120,035 were dissolved in deionized water to a concentration of 1 mM. Peptides were then further diluted in assay buffer (Hank's balanced salt solution with 20 mM HEPES) to add peptide to the cells to reach final concentrations ranging from 10 uM to 1 pM. Peptide was added to cells while fluorescence intensity was measured using a NOVOstar microplate reader purchased from BMG Labtech. Calcium mobilization was measured as relative fluorescence intensity expressed as fold-over basal at 5 seconds and 20 seconds after ligand addition.

### Example 5: Treatment of ARDS

A subject with acute respiratory distress syndrome is treated with a peptide having the sequence of SEQ ID NO:27. The subject's symptoms improve and the inflammation associated with ARDS decreases and oxygenation increases.

### Example 6: Treatment of Thrombosis

A subject with thrombosis is treated with a peptide having the sequence of SEQ ID NO:27. The subject's symptoms improve and the thrombosis decreases and the blood clot and/or the thrombus decreases.

### Example 7: Prevention of Thrombosis

A subject with the risk of developing thrombosis is treated with a peptide having the sequence of SEQ ID NO:27. The subject's risk of developing thrombosis decreases and the thrombosis is prevented or the development thereof is delayed.

### Example 8: Treatment of Platelet Adhesion

A subject with platelet adhesion is treated with a peptide having the sequence of SEQ ID NO:27. The subject's symptoms improve and the platelet adhesion decreases and the blood clot and/or the thrombus decreases.

### Example 9: Prevention of Thrombosis

A subject with the risk of developing thrombosis is treated with a peptide having the sequence of SEQ ID NO:27. The subject's risk of developing platelet adhesion that could have led to a thrombotic event decreases and the platelet adhesion is prevented or the development thereof is delayed.

### Example 10: Treatment of Platelet Aggregation

A subject with platelet aggregation is treated with a peptide having the sequence of SEQ ID NO:27. The subject's symptoms improve and the platelet aggregation decreases and the blood clot and/or the thrombus decreases.

### Example 11: Prevention of Platelet Aggregation

A subject with the risk of developing platelet aggregation is treated with a peptide having the sequence of SEQ ID NO:27. The subject's risk of developing platelet aggregation decreases and the platelet aggregation is prevented or the development thereof is delayed.

### Example 12: Reduction in IAS and FAS D-Dimer Response

D-Dimer formation is elevated in patients with ARDS and COVID. A clinical study was performed to determine the ability of a peptide having the sequence of SEQ ID NO:27 to reduce the effects of D-Dimer formation. Patients diagnosed with COVID-19 infection (SAR-CoV-2) were infused at a rate of 12 mg/hr for up to 7 days of a pharmaceutical composition comprising a peptide having the sequence of SEQ ID NO: 27 or a placebo control. Each group was monitored for an IAS D-Dimer response (>30% reduction) and a FAS D-Dimer response (>30% reduction). In the control group, 2/8 patients had greater than a 30% reduction in the IAS D-Dimer response and 1/7 patients had greater than a 30% reduction in the FAS D-Dimer response. In contrast, the patients treated with a pharmaceutical composition comprising a peptide having the sequence of SEQ ID NO: 27, 5 out of 11 patients had greater than 30% reduction in the IAS D-Dimer response and 4 out of 9 had greater than 30% reduction in the FAS D-Dimer response. Therefore, the data illustrates the effect of a peptide as provided for herein can have on the reduction in the D-Dimer response. It is anticipated that the reduction in the D-Dimer response as measured by these assays will lead to clinical benefit in patients that are infected and/or hospitalized with COVID-19.

The primary endpoint of the clinical study was mean change of D-dimer level from the baseline at day three. D-dimer is a biomarker used to monitor the risk of abnormal clotting throughout the vascular system. In patients with COVID-19, an elevation of D-dimer level in circulation is also known to be an accurate predictor of critical disease progression and death. Among patients treated with a peptide having the sequence of SEQ ID NO:27, 70% (7 of 10) experienced a reduction of D-dimer level in circulation, compared to 27% (3 of 11) of patients on placebo. The peptide having the sequence of SEQ ID NO:27 was associated with a 92% probability of a potential beneficial treatment effect, based on a Bayesian model analysis recommended by the study's Data Monitoring and Safety Committee (DMSC).

Unexpectedly, patients receiving a peptide having the sequence of SEQ ID NO:27 experienced an approximately 12-day reduction in the average length of hospital stay compared to placebo (11.4 vs. 23.3 days), with a median reduction of 4 days (8 vs. 12).

While the embodiments have been described with reference to certain embodiments and examples, those skilled in the art recognize that various modifications may be made to the embodiments provided herein without departing from the spirit and scope thereof.

All of the above U.S. patents, U.S. patent application publications, U.S. patent applications, foreign patents, foreign patent applications and non-patent publications referred to in this specification and/or listed in the Application Data Sheet are incorporated herein by reference in their entirety.

What is claimed is:

1. A method of treating acute respiratory distress syndrome (ARDS) in a subject, the method comprising administering to the subject a peptide or peptide mimetic selected from the group consisting of
a peptide or peptide mimetic comprising the sequence of Xx-Yy-Val-Ww-Zz-Aa-Bb-Cc,
wherein Xx is selected from the group consisting of null, sarcosine, N-methyl-L-alanine, N-methyl-D-alanine, N,N-dimethylglycine, L-aspartic acid, D-aspartic acid, L-glutamic acid, D-glutamic acid, N-methyl-L-aspartic acid, N-methyl-L-glutamic acid, pyrrolid-1-ylacetic acid, and morpholin-4-ylacetic acid;
Yy is selected from the group consisting of L-arginine and L-lysine; Ww is selected from the group consisting of L-isoleucine, glycine, L-tyrosine, O-methyl-L-tyrosine, L-valine, L-phenylalanine, 3-hydroxy-L-tyrosine, 2,6-dimethyl-L-tyrosine, 3-fluoro-L-tyrosine, 4-fluorophenyl-L-alanine, 2,6-difluoro-L-tyrosine, 3-nitro-L-tyrosine, 3,5-dinitro-L-tyrosine, 3,5-dibromo-L-tyrosine, 3-chloro-L-tyrosine, O-allyl-L-tyrosine, and 3,5-diiodo-L-tyrosine;
Zz is selected from the group consisting of L-isoleucine, L-valine, L-tyrosine, L-glutamic acid, L-phenylalanine, L-histidine, L-lysine, L-arginine, O-methyl-L-threonine, D-alanine, and L-norvaline;
Aa is selected from the group consisting of L-histidine, L-histidine-amide, and L-lysine;
Bb is selected from the group consisting of L-proline, L-proline-amide, D-proline, and D-proline-amide; and
Cc is selected from the group consisting of null, L-isoleucine, L-isoleucine-amide, glycine, glycine-amide, L-alanine, L-alanine-amide, D-alanine, D-phenylalanine, L-norvaline;
provided that when Xx is L-Aspartic acid, Cc is not L-phenylalanine; when Xx is sarcosine, Cc is not L-isoleucine; when Ww is glycine, Cc is not glycine; when Xx is sarcosine, and Zz is L-valine, Cc is not L-alanine; and when Xx is sarcosine, Ww is L-tyrosine, and Zz is L-isoleucine, Cc is not L-alanine;
a peptide or peptide mimetic wherein the members of the sequence of the peptide or peptide mimetic maintain their relative positions as they appear in the sequence described in (a), wherein spacers of between 1 and 3 amino acids or amino acid analogues are inserted between one or more of the amino acids or amino acid analogues as described in (a) and wherein the total length of the peptide or peptide mimetic is between 6 and 25 amino acids and/or amino acid analogues; and
a peptide or peptide mimetic that is at least 70% identical to the peptide or peptide mimetics described in (a).

2-3. (canceled)

4. The method of claim 1, wherein Ww is selected from the group consisting of L-tyrosine and 3-hydroxy-L-tyrosine; and Zz is selected from the group consisting of L-isoleucine and L-lysine.

5. The method of claim 4, wherein the peptide or peptide mimetic comprises the sequence of SEQ ID NO: 23.

6. The method of claim 4, wherein the peptide or peptide mimetic comprises the sequence of SEQ ID NO: 27.

7. The method of claim 4, wherein the peptide or peptide mimetic comprises the sequence of SEQ ID NO: 67.

8-19. (canceled)

20. The method of claim 1, wherein the peptide or peptide mimetic is cyclic.

21. The method of claim 1, wherein the peptide or peptide mimetic is dimerized.

22. The method of claim 1, wherein the peptide or peptide mimetic is trimerized.

23. The method of claim 1, wherein the peptide or peptide mimetic is administered to the subject in a pharmaceutical composition comprising the peptide or peptide mimetic and a pharmaceutically acceptable earner.

24. The method of claim 23, wherein the pharmaceutically acceptable carrier is pure sterile water, phosphate buffered saline or an aqueous glucose solution.

25. The method of claim 1, wherein the ARDS is viral induced ARDS.

26. The method of claim 1, wherein the ARDS is induced by an influenza virus or a coronavirus.

27. The method of claim 1, wherein the ARDS is caused by COVID-19 infection.

28-32. (canceled)

33. The method of claim 1, wherein the peptide has a sequence of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85.

34. The method of claim 33, wherein the peptide has a sequence of SEQ ID NO: 27.

35-113. (canceled)

114. A method of reducing a D-Dimer level in circulation in a subject with ARDS or a viral infection, the method comprising administering to the subject a peptide or peptide mimetic selected from the group consisting of
a) a peptide or peptide mimetic comprising the sequence of Sar-Arg-Val-Ww-Zz-His-Pro-Cc,
wherein Ww is selected from the group consisting of L-tyrosine, 3-hydroxy-L-tyrosine; 3-fluoro-L-tyrosine, and 3-chloro-L-tyrosine;
Zz is selected from the group consisting of L-isoleucine, L-valine, L-tyrosine, L-glutamic acid, L-phenylalanine, L-histidine, L-lysine, L-arginine, O-methyl-L-threonine, D-alanine, and L-norvaline; and
Cc is selected from the group consisting of D-alanine and L-alanine;
b) a peptide or peptide mimetic wherein the members of the sequence of the peptide or peptide mimetic maintain their relative positions as they appear in the sequence described in (a), wherein spacers of between 1 and 3 amino acids or amino acid analogues are inserted between one or more of the amino acids or amino acid analogues as described in (a) and wherein the total length of the peptide or peptide mimetic is between 8 and 25 amino acids and/or amino acid analogues; and
c) a peptide or peptide mimetic that is at least 70% identical to the peptide or peptide mimetics described in (a).

115. The method of claim 114, wherein the D-Dimer level is reduced by greater than 10, 20, 30, 40, 50, 60, 70, 80, 90, 95, or 99%.

116-133. (canceled)

134. The method of claim 1, wherein the peptide is administered to the subject at a rate of about 1 mg/hr to about 20 mg/hr, about 5 mg/hr to about 20 mg/hr, about 10 mg/hr to about 20 mg/hr, about 10 mg/hr to about 15 mg/hr, about 8 mg/hr to about 15 mg/hr, about 9 mg/hr to about 15 mg/hr, about 12 mg/hr to about 13 mg/hr, about 1 mg/hr, about 2 mg/hr, about 3 mg/hr, about 4 mg/hr, about 5 mg/hr, about 6 mg/hr, about 7 mg/hr, about 8 mg/hr, about 9 mg/hr, about 10 mg/hr, about 11 mg/hr, about 12 mg/hr, about 13 mg/hr, about 14 mg/hr, about 15 mg/hr, about 16 mg/hr, about 17 mg/hr, about 18 mg/hr, about 19, mg/hr, or about 20 mg/hr.

135. (canceled)

136. A method of reducing a length of hospital stay of a subject with ARDS or a viral infection, the method comprising administering to the subject a peptide or peptide mimetic selected from the group consisting of
a peptide or peptide mimetic comprising the sequence of Sar-Arg-Val-Ww-Zz-His-Pro-Cc,
wherein Ww is selected from the group consisting of L-tyrosine, 3-hydroxy-L-tyrosine; 3-fluoro-L-tyrosine, and 3-chloro-L-tyrosine;
Zz is selected from the group consisting of L-isoleucine, L-valine, L-tyrosine, L-glutamic acid, L-phenylalanine, L-histidine, L-lysine, L-arginine, 0-methyl-L-threonine, D-alanine, and L-norvaline; and
Cc is selected from the group consisting of D-alanine and L-alanine;
a peptide or peptide mimetic wherein the members of the sequence of the peptide or peptide mimetic maintain their relative positions as they appear in the sequence described in (a), wherein spacers of between 1 and 3 amino acids or amino acid analogues are inserted between one or more of the amino acids or amino acid analogues as described in (a) and wherein the total length of the peptide or peptide mimetic is between 8 and 25 amino acids and/or amino acid analogues; and
a peptide or peptide mimetic that is at least 70% identical to the peptide or peptide mimetics described in (a).

137. The method of claim 136, wherein the viral infection is a coronavirus infection.

138-145. (canceled)
